SEQUENCE LISTING (3 sequences)

SEQ ID NO: 1 - DNA, 10807 bp, Zika virus
agttgttgatctgtgtgaatcagactgcgacagttcgagtttgaagcgaaagctagcaacagtatcaacaggttttattttggatttggaaacgagagtttctggtcatgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcaattctagcctttttgagattcacggcaatcaagccatcactgggtctcatcaatagatggggttcagtggggaaaaaagaggctatggaaataataaagaagttcaagaaagatctggctgccatgctgagaataatcaatgctaggaaggagaagaagagacggggcgcagatactagtgtcggaattgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacatgtgtgatgccaccatgagctatgaatgccctatgctggatgagggggtggaaccagatgacgtcgattgttggtgcaacacgacgtcaacttgggttgtgtacggaacctgccatcacaaaaaaggtgaagcacggagatctagaagagctgtgacgctcccctcccattccactaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatattgtcttggaacatggaggttgtgtcaccgtaatggcacaggacaaaccgactgtcgacatagagctggttacaacaacagtcagcaacatggcggaggtaagatcctactgctatgaggcatcaatatcagacatggcttcggacagccgctgcccaacacaaggtgaagcctaccttgacaagcaatcagacactcaatatgtctgcaaaagaacgttagtggacagaggctggggaaatggatgtggactttttggcaaagggagtctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaggttgagataacgcccaattcaccaagagccgaagccaccctggggggttttggaagcctaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtaccgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggtctgaacacaaagaatggatctatttcccttatgtgcttggccttagggggagtgttgatcttcttatccacagccgtctctgctgatgtggggtgctcggtggacttctcaaagaaggagacgagatgcggtacaggggtgttcgtctataacgacgttgaagcctggagggacaggtacaagtaccatcctgactccccccgtagattggcagcagcagtcaagcaagcctgggaagatggtatctgcgggatctcctctgtttcaagaatggaaaacatcatgtggagatcagtagaaggggagctcaacgcaatcctggaagagaatggagttcaactgacggtcgttgtgggatctgtaaaaaaccccatgtggggaggtccacagagattgcccgtgcctgtgaacgagctgccccacggctggaaggcttgggggaaatcgcacttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgttattggaacagctgttaagggaaaggaggctgtacacagtgatctaggctactggattgagagtgagaagaatgacacatggaggctgaagagggcccatctgatcgagatgaaaacatgtgaatggccaaagtcccacacattgtggacagatggaatagaagagagtgatctgatcatacccaagtctttagctgggccactcagccatcacaataccagagagggctacaggacccaaatgaaagggccatggcacagtgaagagcttgaaattcggtttgaggaatgcccaggcactaaggtccacgtggaggaaacatgtggaacaagaggaccatctctgagatcaaccactgcaagcggaagggtgatcgaggaatggtgctgcagggagtgcacaatgcccccactgtcgttccgggctaaagatggctgttggtatggaatggagataaggcccaggaaagaaccagaaagcaacttagtaaggtcaatggtgactgcaggatcaactgatcacatggatcacttctcccttggagtgcttgtgattctgctcatggtgcaggaagggctgaagaagagaatgaccacaaagatcatcataagcacatcaatggcagtgctggtagctatgatcctgggaggattttcaatgagtgacctggctaagcttgcaattttgatgggtgccaccttcgcggaaatgaacactggaggagatgtagctcatctggcgctgatagcggcattcaaagtcagaccagcgttgctggtatctttcatcttcagagctaattggacaccccgtgaaagcatgctgctggccttggcctcgtgtcttttgcaaactgcgatctccgccttggaaggcgacctgatggttctcatcaatggttttgctttggcctggttggcaatacgagcgatggttgttccacgcactgataacatcaccttggcaatcctggctgctctgacaccactggcccggggcacactgcttgtggcgtggagagcaggccttgctacttgcggggggtttatgctcctctctctgaagggaaaaggcagtgtgaagaagaacttaccatttgtcatggccctgggactaaccgctgtgaggctggtcgaccccatcaacgtggtggggctgctgttgctcacaaggagtgggaagcggagctggccccctagcgaagtactcacagctgttggcctgatatgcgcattggctggagggttcgccaaggcagatatagagatggctgggcccatggccgcggtcggtctgctaattgtcagttacgtggtctcaggaaagagtgtggacatgtacattgaaagagcaggtgacatcacatgggaaaaagatgcggaagtcactggaaacagtccccggctcgatgtggcgctagatgagagtggtgatttctccctggtggaggatgacggtccccccatgagagagatcatactcaaggtggtcctgatgaccatctgtggcatgaacccaatagccataccctttgcagctggagcgtggtacgtatacgtgaagactggaaaaaggagtggtgctctatgggatgtgcctgctcccaaggaagtaaaaaagggggagaccacagatggagtgtacagagtaatgactcgtagactgctaggttcaacacaagttggagtgggagttatgcaagagggggtctttcacactatgtggcacgtcacaaaaggatccgcgctgagaagcggtgaagggagacttgatccatactggggagatgtcaagcaggatctggtgtcatactgtggtccatggaagctagatgccgcctgggacgggcacagcgaggtgcagctcttggccgtgccccccggagagagagcgaggaacatccagactctgcccggaatatttaagacaaaggatggggacattggagcggttgcgctggattacccagcaggaacttcaggatctccaatcctagacaagtgtgggagagtgataggactttatggcaatggggtcgtgatcaaaaatgggagttatgttagtgccatcacccaagggaggagggaggaagagactcctgttgagtgcttcgagccttcgatgctgaagaagaagcagctaactgtcttagacttgcatcctggagctgggaaaaccaggagagttcttcccgaaatagtccgtgaagccataaaaacaagactccgtactgtgatcttagctccaaccagggttgtcgctgctgaaatggaggaagcccttagagggcttccagtgcgttatatgacaacagcagtcaatgtcacccactctggaacagaaatcgtcgacttaatgtgccatgccaccttcacttcacgtctactacagccaatcagagtccccaactataatctgtatattatggatgaggcccacttcacagatccctcaagtatagcagcaagaggatacatttcaacaagggttgagatgggcgaggcggctgccatcttcatgaccgccacgccaccaggaacccgtgacgcatttccggactccaactcaccaattatggacaccgaagtggaagtcccagagagagcctggagctcaggctttgattgggtgacggattattctggaaaaacagtttggtttgttccaagcgtgaggaacggcaatgagatcgcagcttgtctgacaaaggctggaaaacgggtcatacagctcagcagaaagacttttgagacagagttccagaaaacaaaacatcaagagtgggactttgtcgtgacaactgacatttcagagatgggcgccaactttaaagctgaccgtgtcatagattccaggagatgcctaaagccggtcatacttgatggcgagagagtcattctggctggacccatgcctgtcacacatgccagcgctgcccagaggagggggcgcataggcaggaatcccaacaaacctggagatgagtatctgtatggaggtgggtgcgcagagactgacgaagaccatgcacactggcttgaagcaagaatgctccttgacaatatttacctccaagatggcctcatagcctcgctctatcgacctgaggccgacaaagtagcagccattgagggagagttcaagcttaggacggagcaaaggaagacctttgtggaactcatgaaaagaggagatcttcctgtttggctggcctatcaggttgcatctgccggaataacctacacagatagaagatggtgctttgatggcacgaccaacaacaccataatggaagacagtgtgccggcagaggtgtggaccagacacggagagaaaagagtgctcaaaccgaggtggatggacgccagagtttgttcagatcatgcggccctgaagtcattcaaggagtttgccgctgggaaaagaggagcggcttttggagtgatggaagccctgggaacactgccaggacacatgacagagagattccaggaagccattgacaacctcgctgtgctcatgcgggcagagactggaagcaggccttacaaagccgcggcggcccaattgccggagaccctagagaccattatgcttttggggttgctgggaacagtctcgctgggaatctttttcgtcttgatgaggaacaagggcatagggaagatgggctttggaatggtgactcttggggccagcgcatggctcatgtggctctcggaaattgagccagccagaattgcatgtgtcctcattgttgtgttcctattgctggtggtgctcatacctgagccagaaaagcaaagatctccccaggacaaccaaatggcaatcatcatcatggtagcagtaggtcttctgggcttgattaccgccaatgaactcggatggttggagagaacaaagagtgacctaagccatctaatgggaaggagagaggagggggcaaccatgggattctcaatggacattgacctgcggccagcctcagcttgggccatctatgctgccttgacaactttcattaccccagccgtccaacatgcagtgaccacttcatacaacaactactccttaatggcgatggccacgcaagctggagtgttgtttggtatgggcaaagggatgccattctacgcatgggactttggagtcccgctgctaatgataggttgctactcacaattaacgcccctgaccctaatagtggccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatggcagtagccgtctccagcgccatactgtcgcggaccgcctgggggtggggggaggctggggccctgatcacagccgcaacttccactttgtgggaaggctctccgaacaagtactggaactcctctacagccacttcactgtgtaacatttttaggggaagttacttggctggagcttctctaatctacacagtgacaagaaacgctggcttggtcaagagacgtgggggtggaacaggagagaccctgggagagaaatggaaggcccgcttgaaccagatgtcggccctggagttctactcctacaaaaagtcaggcatcaccgaggtgtgcagagaagaggcccgccgcgccctcaaggacggtgtggcaacgggaggccatgctgtgtcccgaggaagtgcaaagctgagatggttggtggagcggggatacctgcagccctatggaaaggtcattgatcttggatgtggcagagggggctggagttactacgccgccaccatccgcaaagttcaagaagtgaaaggatacacaaaaggaggccctggtcatgaagaacccgtgttggtgcaaagctatgggtggaacatagtccgtcttaagagtggggtggacgtctttcatatggcggctgagccgtgtgacacgttgctgtgtgacataggtgagtcatcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctcctcttggggcgcatggacgggcctaggaggccagtgaaatatgaggaggatgtgaatctcggctctggcacgcgggctgtggtaagctgcgctgaagctcccaacatgaagatcattggtaaccgcattgaaaggatccgcagtgagcacgcggaaacgtggttctttgacgagaaccacccatataggacatgggcttaccatggaagctatgaggcccccacacaagggtcagcgtcctctctaataaacggggttgtcaggctcctgtcaaaaccctgggatgtggtgactggagtcacaggaatagccatgaccgacaccacaccgtatggtcagcaaagagttttcaaggaaaaagtggacactagggtgccagacccccaagaaggtactcgtcaggttatgagcatggtctcttcctggttgtggaaagagctaggcaaacacaaacggccacgagtctgtaccaaagaagagttcatcaacaaggttcgtagcaatgcagcattaggggcaatatttgaagaggaaaaagagtggaagactgcagtggaagctgtgaacgatccaaggttctgggctctagtggacaaggaaagagagcaccacctgagaggagagtgccagagttgtgtgtacaacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgcataccaggaggaaggatgtatgcagatgacactgctggctgggacacccgcatcagcaggtttgatctggagaatgaagctctaatcaccaaccaaatggagaaagggcacagggccttggcattggccataatcaagtacacataccaaaacaaagtggtaaaggtccttagaccagctgaaaaagggaaaacagttatggacattatttcgagacaagaccaaagggggagcggacaagttgtcacttacgctcttaacacatttaccaacctagtggtgcaactcattcggaatatggaggctgaggaagtcctagagatgcaagacttgtggctgctgcggaggtcagagaaagtgaccaactggttgcagagcaacggatgggataggctcaaacgaatggcagtcagtggagatgattgcgttgtgaagccaattgatgataggtttgcacatgccctcaggttcttgaatgatatgggaaaagttaggaaggacacacaagagtggaaaccctcaactggatgggacaactgggaagaagttccgttttgctcccaccacttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaacacagtcaacatggtgcgcaggatcataggtgatgaagaaaagtacatggactacctatccacccaagttcgctacttgggtgaagaagggtctacacctggagtgctgtaagcaccaatcttaatgttgtcaggcctgctagtcagccacagcttggggaaagctgtgcagcctgtgacccccccaggagaagctgggaaaccaagcctatagtcaggccgagaacgccatggcacggaagaagccatgctgcctgtgagcccctcagaggacactgagtcaaaaaaccccacgcgcttggaggcgcaggatgggaaaagaaggtggcgaccttccccacccttcaatctggggcctgaactggagatcagctgtggatctccagaagagggactagtggttagaggagaccccccggaaaacgcaaaacagcatattgacgctgggaaagaccagagactccatgagtttccaccacgctggccgccaggcacagatcgccgaatagcggcggccggtgtggggaaatccatgggtct

SEQ ID NO: 2 - protein, 3423 aa, Zika virus
MKNPKKKSGGFRIVNMLKRGVARVSPFGGLKRLPAGLLLGHGPIRMVLAILAFLRFTAIKPSLGLINRWGSVGKKEAMEIIKKFKKDLAAMLRIINARKEKKRRGADTSVGIVGLLLTTAMAAEVTRRGSAYYMYLDRNDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDIVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSADVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWGGPQRLPVPVNELPHGWKAWGKSHFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTAGSTDHMDHFSLGVLVILLMVQEGLKKRMTTKIIISTSMAVLVAMILGGFSMSDLAKLAILMGATFAEMNTGGDVAHLALIAAFKVRPALLVSFIFRANWTPRESMLLALASCLLQTAISALEGDLMVLINGFALAWLAIRAMVVPRTDNITLAILAALTPLARGTLLVAWRAGLATCGGFMLLSLKGKGSVKKNLPFVMALGLTAVRLVDPINVVGLLLLTRSGKRSWPPSEVLTAVGLICALAGGFAKADIEMAGPMAAVGLLIVSYVVSGKSVDMYIERAGDITWEKDAEVTGNSPRLDVALDESGDFSLVEDDGPPMREIILKVVLMTICGMNPIAIPFAAGAWYVYVKTGKRSGALWDVPAPKEVKKGETTDGVYRVMTRRLLGSTQVGVGVMQEGVFHTMWHVTKGSALRSGEGRLDPYWGDVKQDLVSYCGPWKLDAAWDGHSEVQLLAVPPGERARNIQTLPGIFKTKDGDIGAVALDYPAGTSGSPILDKCGRVIGLYGNGVVIKNGSYVSAITQGRREEETPVECFEPSMLKKKQLTVLDLHPGAGKTRRVLPEIVREAIKTRLRTVILAPTRVVAAEMEEALRGLPVRYMTTAVNVTHSGTEIVDLMCHATFTSRLLQPIRVPNYNLYIMDEAHFTDPSSIAARGYISTRVEMGEAAAIFMTATPPGTRDAFPDSNSPIMDTEVEVPERAWSSGFDWVTDYSGKTVWFVPSVRNGNEIAACLTKAGKRVIQLSRKTFETEFQKTKHQEWDFVVTTDISEMGANFKADRVIDSRRCLKPVILDGERVILAGPMPVTHASAAQRRGRIGRNPNKPGDEYLYGGGCAETDEDHAHWLEARMLLDNIYLQDGLIASLYRPEADKVAAIEGEFKLRTEQRKTFVELMKRGDLPVWLAYQVASAGITYTDRRWCFDGTTNNTIMEDSVPAEVWTRHGEKRVLKPRWMDARVCSDHAALKSFKEFAAGKRGAAFGVMEALGTLPGHMTERFQEAIDNLAVLMRAETGSRPYKAAAAQLPETLETIMLLGLLGTVSLGIFFVLMRNKGIGKMGFGMVTLGASAWLMWLSEIEPARIACVLIVVFLLLVVLIPEPEKQRSPQDNQMAIIIMVAVGLLGLITANELGWLERTKSDLSHLMGRREEGATMGFSMDIDLRPASAWAIYAALTTFITPAVQHAVTTSYNNYSLMAMATQAGVLFGMGKGMPFYAWDFGVPLLMIGCYSQLTPLTLIVAIILLVAHYMYLIPGLQAAAARAAQKRTAAGIMKNPVVDGIVVTDIDTMTIDPQVEKKMGQVLLMAVAVSSAILSRTAWGWGEAGALITAATSTLWEGSPNKYWNSSTATSLCNIFRGSYLAGASLIYTVTRNAGLVKRRGGGTGETLGEKWKARLNQMSALEFYSYKKSGITEVCREEARRALKDGVATGGHAVSRGSAKLRWLVERGYLQPYGKVIDLGCGRGGWSYYAATIRKVQEVKGYTKGGPGHEEPVLVQSYGWNIVRLKSGVDVFHMAAEPCDTLLCDIGESSSSPEVEEARTLRVLSMVGDWLEKRPGAFCIKVLCPYTSTMMETLERLQRRYGGGLVRVPLSRNSTHEMYWVSGAKSNTIKSVSTTSQLLLGRMDGPRRPVKYEEDVNLGSGTRAVVSCAEAPNMKIIGNRIERIRSEHAETWFFDENHPYRTWAYHGSYEAPTQGSASSLINGVVRLLSKPWDVVTGVTGIAMTDTTPYGQQRVFKEKVDTRVPDPQEGTRQVMSMVSSWLWKELGKHKRPRVCTKEEFINKVRSNAALGAIFEEEKEWKTAVEAVNDPRFWALVDKEREHHLRGECQSCVYNMMGKREKKQGEFGKAKGSRAIWYMWLGARFLEFEALGFLNEDHWMGRENSGGGVEGLGLQRLGYVLEEMSRIPGGRMYADDTAGWDTRISRFDLENEALITNQMEKGHRALALAIIKYTYQNKVVKVLRPAEKGKTVMDIISRQDQRGSGQVVTYALNTFTNLVVQLIRNMEAEEVLEMQDLWLLRRSEKVTNWLQSNGWDRLKRMAVSGDDCVVKPIDDRFAHALRFLNDMGKVRKDTQEWKPSTGWDNWEEVPFCSHHFNKLHLKDGRSIVVPCRHQDELIGRARVSPGAGWSIRETACLAKSYAQMWQLLYFHRRDLRLMANAICSSVPVDWVPTGRTTWSIHGKGEWMTTEDMLVVWNRVWIEENDHMEDKTPVTKWTDIPYLGKREDLWCGSLIGHRPRTTWAENIKNTVNMVRRIIGDEEKYMDYLSTQVRYLGEEGSTPGVL

SEQ ID NO: 3 - protein, 11 aa, Artificial Sequence
  Description of Artificial Sequence Synthetic peptide
KLKLLLLLKLK

(19) United States
(12) Patent Application Publication
(10) Pub. No.: US 2020/0330581 A1
(43) Pub. Date: Oct. 22, 2020

(54) ONCOLYTIC CANCER IMMUNOTHERAPIES AND METHODS OF USE

(71) Applicant: Keith BLACK, Los Angeles, CA (US)

(72) Inventor: Keith BLACK, Los Angeles, CA (US)

(21) Appl. No.: 16/754,108

(22) PCT Filed: Oct. 8, 2018
(86) PCT No.: PCT/US2018/054857

§ 371 Date: Apr. 6, 2020

Related U.S. Application Data
(60) Provisional application No. 62/569,989, filed on Oct. 9, 2017.

(51) Int. Cl. A61K 39/12; A61K 35/768; C12N 7/00; A61K 31/7052; A61K 45/06; A61P 35/00

(52) U.S. Cl. CPC A61K 39/12; A61K 35/768; C12N 7/00; A61K 2039/5256; A61K 45/06; A61P 35/00; C12N 2770/24132; A61K 31/7052

(57) ABSTRACT

Disclosed herein are methods of treating a brain cancer comprising administering to a subject in need thereof a viral composition comprising a flavivirus or portion of a flavivirus wherein the flavivirus is engineered to comprise a heterologous nucleic acid sequence encoding a suicide gene. In some embodiments, the brain cancer is selected from the group consisting of astrocytoma, oligodendroglioma, ependymoma, meningioma, schwannoma, craniopharyngioma, germinoma, and pineocytoma. In some embodiments the flavivirus comprises Zika virus, spondweni virus, kedougous virus, or a combination thereof. In some embodiments, the flavivirus comprises Zika virus. In some embodiments, the suicide gene encodes a protein that converts a prodrug into a cytotoxic agent. In some embodiments, the viral composition is administered in combination with the immunostimulatory agent.

### CROSS-REFERENCE

This application claims the priority benefit of U.S. provisional application 62/569,989 filed Oct. 9, 2017, which is hereby incorporated herein in its entirety for all purposes.

### SEQUENCE LISTING

The instant application contains a Sequence Listing which has been submitted electronically in ASCII format and is hereby incorporated by reference in its entirety. Said ASCII copy, created on Oct. 4, 2018, is named 50014-704_601_SL.txt and is 44,889 bytes in size.

### BACKGROUND OF THE INVENTION

Cancers of the brain and nervous system are among the most difficult to treat, with treatment primarily consisting of surgical removal and radiation therapy, and limited chemotherapy options due to the difficulties of passing the brain-blood barrier. Unfortunately, new therapeutics have failed to improve overall survival or lead to a useful clinical response. Thus there is an unmet need for a new therapeutic approach that can impact the course of the disease.

### SUMMARY OF THE INVENTION

One therapeutic approach for treating cancer is immunotherapy, where the immune system is exploited to target and/or destroy cancer cells. However some immunotherapies, such as chimeric antigen receptor (CAR) T-cell therapy, have not been effective for treating solid tumors. A significant hurdle for applying CAR T-cell therapy to solid tumors is the limited ability to traffic adoptively transferred T-cells to tumor sites.

Disclosed herein are methods of treating a brain cancer comprising administering to a subject in need thereof a viral composition comprising a flavivirus or portion of a flavivirus wherein the flavivirus is engineered to comprise a heterologous nucleic acid sequence encoding a suicide gene. In some embodiments, the brain cancer is selected from the group consisting of astrocytoma, oligodendroglioma, ependymoma, meningioma, schwannoma, craniopharyngioma, germinoma, and pineocytoma. In some embodiments, the flavivirus comprises Zika virus, spondweni virus, kedougous virus, or a combination thereof. In some embodiments, the flavivirus comprises Zika virus. In some embodiments, the flavivirus comprises a nucleic acid sequence encoding for a capsid protein of the flavivirus, a membrane protein of the flavivirus, an envelope protein of the flavivirus, a nonstructural (NS) protein of the flavivirus, or a combination thereof. In some embodiments, the suicide gene is expressible under an inducible promoter. In some embodiments, the inducible promoter is cytomegalovirus (CMV). In some embodiments, the suicide gene encodes a protein that converts a prodrug into a cytotoxic agent. In some embodiments, the protein that converts a prodrug into the cytotoxic agent has enzymatic activity selected from the group consisting of thymidine kinase activity, cytosine deaminase activity, purine nucleoside phosphorylase activity, uracil phosphoribosyl transferase activity, and thymidylate kinase activity. In some embodiments, the prodrug is selected from the group consisting of ganciclovir, ganciclovir elaidic acid ester, penciclovir, acyclovir, valacyclovir, (E)-5-(2-bromovinyl)-2′deoxyuridine, zidovudine, 2′-exo-methanocarbathymidine, 5-fluorocytosine, 6-methylpurine deoxyriboside, fludarabine, 5-fluorocytosine, 5-fluorouracil, and azidothymidine. In some embodiments, the prodrug is administered to the subject. In some embodiments, the viral composition promotes an immune response to the brain cancer. In some embodiments, the viral composition causes preferential mitotic impairment to a cancer cell as compared to a non-cancer cell, adult stem cell, or post-mitotic cell. In some embodiments, the subject is administered an antiviral agent configured to kill the flavivirus. In some embodiments, the antiviral agent comprises a nucleoside analog. In some embodiments, the nucleoside analog comprises sofosbuvir, 2′-C-methyladenosine, or a combination thereof. In some embodiments, the subject is administered an immunostimulatory agent. In some embodiments, the viral composition is administered in combination with the immunostimulatory agent. In some embodiments, the viral composition comprises the immunostimulatory agent. In some embodiments, the flavivirus or portion of the flavivirus targets the immunostimulatory agent to the cancer. In some embodiments, the viral composition and the immunostimulatory agent provide a synergistic immune response to the cancer. In some embodiments, the viral composition and immunostimulatory agent are administered in a single composition. In some embodiments, the viral composition and immunostimulatory agent are administered as separate compositions. In some embodiments, the immunostimulatory agent comprises a Toll-like receptor (TLR) agonist. In some embodiments, the Toll-like receptor agonist comprises lipopolysaccharide (LPS), imiquimod (TLR7 agonist), rintatolimod, resiquimod, or a combination thereof. In some embodiments, the immunostimulatory agent comprises granulocyte-macrophage colony-stimulating factor (GMCSF), heat shock protein (Hsp) 70, Hsp90, Hsp110, CpG oligonucleotide, or a combination thereof. In some embodiments, the immunostimulatory agent comprises an adjuvant.

Disclosed herein are pharmaceutical compositions comprising an effective amount of a flavivirus or portion of a flavivirus wherein the flavivirus is engineered to comprise a heterologous nucleic acid sequence encoding a suicide gene and a pharmaceutically acceptable excipient. In some embodiments, the flavivirus comprises Zika virus. In some embodiments, the suicide gene encodes a protein that converts a prodrug into a cytotoxic agent. In some embodiments, the protein that converts a prodrug into the cytotoxic agent has enzymatic activity selected from the group consisting of thymidine kinase activity, cytosine deaminase activity, purine nucleoside phosphorylase activity, uracil phosphoribosyl transferase activity, and thymidylate kinase activity.

In one aspect, provided herein are viral compositions for use in cancer immunotherapy to treat cancer, such as a solid tumor. In an exemplary embodiment, the composition comprises a virus having tropism for a cancer cell such that when it is administered to a cancer patient it localizes to the cancer cells and produces danger signals that elicit an immune response at the cancer site. Some embodiments involve vaccination strategies to further mediate an immune system response to the viral composition.

In one aspect, provided herein is a method of promoting an immune response to cancer comprising administering to a subject in need thereof a viral composition comprising a flavivirus or a portion of the flavivirus. In some embodiments, the flavivirus or portion of the flavivirus comprises a danger signal that promotes the immune response to the cancer. In some embodiments, the flavivirus has tropism for cancer cells. In some embodiments, the flavivirus has tropism for dividing cells. In some embodiments, the viral composition targets the immune response to the cancer in the subject. In some embodiments, the viral composition causes mitotic impairment, cancer cell death, or both mitotic impairment and cancer cell death. In some embodiments, the viral composition causes preferential mitotic impairment to a cancer cell as compared to a non-cancer cell, adult stem cell, and/or post-mitotic cell. In some embodiments, the cancer comprises a solid tumor. In some embodiments, the cancer comprises a brain cancer. In some embodiments, the brain cancer is astrocytoma, oligodendroglioma, ependymoma, meningioma, schwannoma, craniopharyngioma, germinoma, pineocytoma, or a combination thereof. In some embodiments, administering comprises intraperitoneal injection, intracranial injection, intratumoral injection, systemic administration, parenteral administration, intravenous injection, intrathecal administration, intravenous administration, or a combination thereof.

In some embodiments, the flavivirus comprises Zika virus, spondweni virus, kedougous virus, or a combination thereof. In some embodiments, the portion of the flavivirus comprises at least about 10, 20, 30, 40, 50, 80, 100, 150, or 200 nucleic acids having at least about 90% sequence identity to a nucleic acid sequence of the flavivirus. In some embodiments, the viral composition comprises a nucleic acid sequence at least about 20, 50, 100, 150, 200, 300, or 500 nucleic acids in length, having at least about 70%, 75%, 80%, 85%, 90%, or 95% identity to a nucleic acid sequence of a Zika virus. In some embodiments, the viral composition comprises a nucleic acid sequence at least about 20, 50, 100, 150, 200, 300, or 500 nucleic acids in length, having at least about 70%, 75%, 80%, 85%, 90%, or 95% identity to a sequence of SEQ ID NO: 1. In some embodiments, the flavivirus or portion of the flavivirus comprises a nucleic acid sequence encoding for a capsid protein of the flavivirus, a membrane protein of the flavivirus, an envelope protein of the flavivirus, a nonstructural (NS) protein of the flavivirus, or a combination thereof. In some embodiments, the capsid protein comprises an amino acid sequence at least about 70%, 75%, 80%, 85%, 90%, or 95% identical to the amino acid sequence 1-122 of SEQ ID NO: 2. In some embodiments, the membrane protein comprises an amino acid sequence at least about 70%, 75%, 80%, 85%, 90%, or 95% identical to the amino acid sequence 216-290 of SEQ ID NO: 2. In some embodiments, the envelope protein comprises an amino acid sequence at least about 70%, 75%, 80%, 85%, 90%, or 95% identical to the amino acid sequence 795-1146 of SEQ ID NO: 2. In some embodiments, the NS protein comprises an amino acid sequence at least about 70%, 75%, 80%, 85%, 90%, or 95% identical to the amino acid sequence 1147-1372 of SEQ ID NO: 2. In some embodiments, the NS protein comprises an amino acid sequence at least about 70%, 75%, 80%, 85%, 90%, or 95% identical to the amino acid sequence 1373-1502 of SEQ ID NO: 2. In some embodiments, the NS protein comprises an amino acid sequence at least about 70%, 75%, 80%, 85%, 90%, or 95% identical to the amino acid sequence 1503-2119 of SEQ ID NO: 2. In some embodiments, the NS protein comprises an amino acid sequence at least about 70%, 75%, 80%, 85%, 90%, or 95% identical to the amino acid sequence 2120-2246 of SEQ ID NO: 2. In some embodiments, the NS protein comprises an amino acid sequence at least about 70%, 75%, 80%, 85%, 90%, or 95% identical to the amino acid sequence 2247-2269 of SEQ ID NO: 2. In some embodiments, the NS protein comprises an amino acid sequence at least about 70%, 75%, 80%, 85%, 90%, or 95% identical to the amino acid sequence 2521-3423 of SEQ ID NO: 2. In some embodiments, the NS protein comprises NS1, NS2A, NS2B, NS3, NS4A, NS4B, NS5, or a portion or combination thereof.

In some embodiments, the method further comprises administering to the subject an antiviral agent configured to kill the flavivirus. In some embodiments, the antiviral agent comprises a nucleoside analog. In some embodiments, the nucleoside analog comprises sofosbuvir, 2′-C-methyladenosine, or a combination thereof.

In some embodiments, the subject is administered an immunostimulatory agent. In some embodiments, the viral composition is administered in combination with the immunostimulatory agent. In some embodiments, the viral composition comprises the immunostimulatory agent. In some embodiments, the flavivirus or portion of the flavivirus targets the immunostimulatory agent to the cancer. In some embodiments, the viral composition and the immunostimulatory agent provide a synergistic effect on the immune response to the cancer. In some embodiments, the viral composition and immunostimulatory agent are administered in a single composition. In some embodiments, the viral composition and immunostimulatory agent are administered as separate compositions. In some embodiments, the immunostimulatory agent comprises a Toll-like receptor (TLR) agonist. In some embodiments, the Toll-like receptor agonist comprises lipopolysaccharide (LPS), imiquimod (TLR7 agonist), rintatolimod, resiquimod, or a combination thereof. In some embodiments, the immunostimulatory agent comprises granulocyte-macrophage colony-stimulating factor (GMCSF), heat shock protein (Hsp) 70, Hsp90, Hsp110, CpG oligonucleotide, or a combination thereof.

In some embodiments, the immunostimulatory agent comprises an adjuvant. In some embodiments, the adjuvant comprises an aluminum salt. In some embodiments, the aluminum salt comprises aluminum phosphate, aluminum hydroxide, aluminum potassium sulfate, or a combination thereof. In some embodiments, the adjuvant comprises an oil-in-water emulsion. In some embodiments, the oil-in-water emulsion comprises squalene, polysorbate 80 (Tween 80), sorbitan trioleate, alpha-tocopherol, or a combination thereof. In some embodiments, the oil-in-water emulsion comprises MF59. In some embodiments, the oil-in-water emulsion comprises AS03 (Adjuvant System 03). In some embodiments, the adjuvant comprises a liposome. In some embodiments, the adjuvant comprises a Toll-like receptor agonist. In some embodiments, the adjuvant comprises GLA-SE (glucopyranosyl lipid adjuvant formulated in a stable emulsion). In some embodiments, the adjuvant comprises a synthetic oligodeoxynucleotide (ODN1a). In some embodiments, the adjuvant comprises a peptide comprising KLKL(5)KLK (SEQ ID NO: 3). In some embodiments, the adjuvant comprises IC31®. In some embodiments, the adjuvant comprises dimethyldioctadecyl-ammonium (DDA). In some embodiments, the adjuvant comprises trehalose 6,6-dibehenate (TDB). In some embodiments, the adjuvant comprises CAF01. In some embodiments, the adjuvant comprises monophosphoryl lipid A.

In some embodiments, the subject is treated with an additional cancer therapy. In some embodiments, the viral composition is administered in combination with the additional cancer therapy. In some embodiments, the viral composition comprises the additional cancer therapy. In some embodiments, the flavivirus or portion of the flavivirus targets the additional cancer therapy to the cancer. In some embodiments, the viral composition and the additional cancer therapy provide a synergistic effect on the immune response to the cancer. In some embodiments, the viral composition and additional cancer therapy are administered in a single composition. In some embodiments, the viral composition and additional cancer therapy are administered as separate compositions. In some embodiments, the cancer therapy comprises an immune checkpoint inhibitor. In some embodiments, the immune checkpoint inhibitor comprises an anti-CTLA-4 antibody, an anti-PD-1 antibody, an anti-PD-1L antibody, or a combination thereof. In some embodiments, the anti-CTLA-4 antibody is ipilimumab or an antibody that binds to the same epitope as ipilimumab. In some embodiments, the anti-PD-1 antibody is pembrolizumab or an antibody that binds to the same epitope as pembrolizumab. In some embodiments, the anti-PD-1 antibody is nivolumab or an antibody that binds to the same epitope as nivolumab. In some embodiments, the anti-PD-1 antibody is BGB-A317 or an antibody that binds to the same epitope as BGB-A317. In some embodiments, the anti-PD-1L antibody is avelumab or an antibody that binds to the same epitope as avelumab. In some embodiments, the anti-PD-1L antibody is atezolizumab or an antibody that binds to the same epitope as atezolizumab. In some embodiments, the anti-PD-1L antibody is durvalumab or an antibody that binds to the same epitope as durvalumab. In some embodiments, the cancer therapy comprises adoptive T-cell therapy. In some embodiments, the cancer therapy comprises an anti-CD47 antibody. In some embodiments, the cancer therapy comprises administration of a cytokine. In some embodiments, the cytokine is an interferon. In some embodiments, the cytokine is an interleukin. In some embodiments, the cancer therapy comprises adoptive cell transfer. In some embodiments, the cancer therapy comprises chimeric antigen receptor (CAR) T-cell therapy. In some embodiments, the cancer therapy comprises radiation therapy. In some embodiments, the cancer therapy comprises chemotherapy. In some embodiments, the chemotherapy induces immunogenic cancer cell death. In some embodiments, the chemotherapy comprises doxorubicin and/or docetaxel. In some embodiments, the cancer therapy comprises surgery.

In some embodiments, the flavivirus or portion of the flavivirus is genetically engineered. In some embodiments, the genetically engineered virus binds to a tumor-specific antigen of a cell from the cancer. In some embodiments, the tumor-specific antigen is EGFR or a portion or derivative thereof. In some embodiments, the portion or derivative of EGFR comprises EGFRvIII. In some embodiments, the genetically engineered virus binds to a tumor-associated antigen of a cell from the cancer. In some embodiments, the cell from the cancer is a brain cancer cell. In some embodiments, the engineered virus expresses IL-12. In some embodiments, the engineered virus expresses GMCSF. In some embodiments, the flavivirus is engineered to comprise a suicide gene expressible under an inducible promoter.

In some embodiments, the method comprises administering to the subject the promoter or an activator thereof to express the suicide gene, thereby killing the flavivirus. In some embodiments, the viral composition comprises a recombinant binding domain having specificity for a cancer cell marker. In some embodiments, the flavivirus is engineered for selective proliferation in the cancer cell. In some embodiments, the flavivirus comprises a genetic element of a Zika virus genome that is not found in a dengue virus genome.

In another aspect, provided herein is a combination comprising a flavivirus or a portion of the flavivirus, and an immunostimulatory agent. In some embodiments, the flavivirus or portion of the flavivirus and the immunostimulatory agent are provided in a single composition. In some embodiments, the flavivirus or portion of the flavivirus and the immunostimulatory agent are provided in separate compositions. In some embodiments, the immunostimulatory agent comprises a Toll-like receptor (TLR) agonist. In some embodiments, the Toll-like receptor agonist comprises lipopolysaccharide (LPS), imiquimod (TLR7 agonist), rintatolimod, resiquimod, or a combination thereof. In some embodiments, the immunostimulatory agent comprises granulocyte-macrophage colony-stimulating factor (GMCSF), heat shock protein (Hsp) 70, Hsp90, Hsp110, CpG oligonucleotide, or a combination thereof.

In some embodiments, the immunostimulatory agent comprises an adjuvant. In some embodiments, the adjuvant comprises an aluminum salt. In some embodiments, the aluminum salt comprises aluminum phosphate, aluminum hydroxide, aluminum potassium sulfate, or a combination thereof. In some embodiments, the adjuvant comprises an oil-in-water emulsion. In some embodiments, the oil-in-water emulsion comprises squalene, polysorbate 80 (Tween 80), sorbitan trioleate, alpha-tocopherol, or a combination thereof. In some embodiments, the oil-in-water emulsion comprises MF59. In some embodiments, the oil-in-water emulsion comprises AS03 (Adjuvant System 03). In some embodiments, the adjuvant comprises a liposome. In some embodiments, the adjuvant comprises a Toll-like receptor agonist. In some embodiments, the adjuvant comprises GLA-SE (glucopyranosyl lipid adjuvant formulated in a stable emulsion). In some embodiments, the adjuvant comprises a synthetic oligodeoxynucleotide (ODN1a). In some embodiments, the adjuvant comprises a peptide comprising KLKL(5)KLK (SEQ ID NO: 3). In some embodiments, the adjuvant comprises IC31®. In some embodiments, the adjuvant comprises dimethyldioctadecyl-ammonium (DDA). In some embodiments, the adjuvant comprises trehalose 6,6-dibehenate (TDB). In some embodiments, the adjuvant comprises CAF01. In some embodiments, the adjuvant comprises monophosphoryl lipid A.

In some embodiments, provided is a method of treating cancer comprising administering the combination described herein to a subject in need thereof. In some embodiments, the flavivirus or portion of the flavivirus targets the immunostimulatory agent to the cancer. In some embodiments, the flavivirus or the portion of the flavivirus, and the immunostimulatory agent provide a synergistic effect on an immune response to the cancer. In some embodiments, the flavivirus or portion of the flavivirus and the immunostimulatory agent are administered in a single composition. In some embodiments, the flavivirus or portion of the flavivirus and the immunostimulatory agent are administered as separate compositions.

### DETAILED DESCRIPTION OF THE INVENTION

In one aspect, viral compositions are provided for the treatment of cancer. As a non-limiting example, the composition comprises a virus having tropism for dividing cells, such as a Zika virus or cytomegalovirus. Another aspect of this disclosure relates to methods of cancer treatment comprising administering the viral compositions provided herein. In some cases where the viral composition is administered as a cancer immunotherapy, the composition further comprises an immunostimulatory agent and/or adjuvant.

Before the present methods and compositions are described, it is to be understood that this disclosure is not limited to a particular method or composition described, and as such may vary. Unless defined otherwise, all technical and scientific terms used herein have the same meaning as commonly understood by one of ordinary skill in the art. All publications mentioned herein are incorporated by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible.

Where a range of values is provided, unless otherwise indicated, each intervening value to the tenth of the unit of the lower limit between the upper and lower limits of that range is also specifically disclosed. Each smaller range between any stated value or intervening value in a stated range is encompassed herein. The upper and lower limits of these smaller ranges may independently be included or excluded in the range, and each range where either, neither or both limits are included in the smaller ranges is also encompassed herein, subject to any specifically excluded limit in the stated range. Where the stated range includes one or both of the limits, ranges excluding either or both of those included limits are also included.

As used herein and in the appended claims, the singular forms “a”, “an”, and “the” include plural referents unless the context clearly dictates otherwise. Thus, for example, reference to “a virus” includes a plurality of such virus. As used herein, “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

### Viral Compositions

In one aspect, provided herein are viral compositions comprising viruses or portions thereof, which may be selective for cells undergoing cell division and/or cancerous cells, and as such are useful for killing said cancerous cells, stimulating and/or enhancing an immune response to the cancerous cells, and/or inhibiting the growth of a tumor. The virus may be a clinical isolate of a virus, a clone, recombinant, or engineered virus derived therefrom. In various embodiments, the viral composition comprises one or more additional agents, including, but not limited to, an immunostimulatory agent and/or an adjuvant. Some embodiments of the viral compositions comprise a combination of a virus or portion thereof with an additional cancer therapy, such as chemotherapy, surgery, immunotherapy, or a combination thereof.

For compositions comprising a portion of a virus, the virus portion includes one or more regions of a nucleic acid and/or protein of the virus. Non-limiting examples of viral proteins include capsid proteins, membrane proteins, non-structural proteins, and envelope proteins. Non-limiting examples of viral nucleic acids include protein coding sequences and non-protein coding sequences. In some cases, a portion of a virus includes at least about 10, 20, 30, 40, 50, 80, 100, 150, or 200 nucleic acids having at least about 90% sequence identity to a nucleic acid sequence of the virus. In some cases, a portion of a virus includes a protein comprising at least about 10, 20, 30, 40, 50, 80, 100, 150, or 200 amino acids having at least about 90% sequence identity to an amino acid sequence of the virus. In some cases, a portion of a virus includes a protein encoded by a nucleic acid sequence comprising at least about 10, 20, 30, 40, 50, 80, 100, 150, or 200 nucleic acids having at least about 90% sequence identity to a nucleic acid sequence of the virus.

In some embodiments, the virus is a recombinant or engineered virus. A non-limiting example of an engineered virus is one comprising a nucleic acid sequence that is derived from a viral genome, for example, by mutation, insertion, and/or deletion of one or more nucleobases. An insertion includes a nucleic acid sequence encoding for one or more proteins. An insertion also includes a promoter, such as a promoter inducible in mammalian cells. A deletion includes removal of a coding sequence that allows for replication of the virus. Accordingly, in some cases an engineered virus indicates that the virus from which the engineered virus is derived has been modified by the introduction of a heterologous nucleic acid or protein, or the alteration of a native nucleic acid sequence. As used herein, viruses include both native and engineered viruses, as well as viruses comprises portions of a native or engineered virus.

One method of generating a recombinant virus involves introducing a viral genome or portion thereof into a cell in whole or in part, for example, in two or more fragments that assemble into the desired viral genome or portion thereof within the cell. A non-limiting method comprises cloning and amplifying nucleic acid fragments covering the genome of a virus, or portion thereof. A promoter sequence, such as a cytomegalovirus (CMV) promoter may be inserted at the end of the first fragment. For viruses having a heterologous nucleic acid sequence, this sequence is fused into or adjoining one of the fragments. In one method to produce a virus, amplified fragments are introduced into cells, for example, by electroporation, and the cells are then transferred into growth medium. Cell supernatants are then recovered and stored or used to infect cells from which clarified virus is obtainable from culture supernatants.

Any suitable method is useful for generating genetic modifications in a virus to generate an engineered virus, including mutagenesis, polymerase chain reaction, homologous recombination, or any other genetic engineering technique available in the art. Mutagenesis includes modification of a nucleotide sequence, a single gene, or blocks of genes, and involves removal, addition and/or substitution of a single or plurality of nucleotide bases. In some cases, genetic modification comprises use of genetic recombination techniques to delete or replace at least part of native viral sequence. For example, a polynucleotide replaces part or all of a region of native viral sequence of interest. Alternatively or additionally, a polynucleotide is inserted into the native viral sequence. This inserted polynucleotide may be functional and encode, e.g., a suicide protein, therapeutic agent, and/or reporter protein. Exemplary non-limiting polynucleotides encoding for reporter proteins include green fluorescent protein, enhanced green fluorescent protein, beta-galactosidase, luciferase, and HSV-tk.

In some embodiments, a virus comprises one or more molecules that impart to the virus an enhanced level of tumor cell specificity. In this way, the virus is targeted to specific tumor types using tumor cell-specific molecules.

In some embodiments, a virus comprises a nucleic acid sequence encoding for a polypeptide that is heterologous to the virus, where the polypeptide is expressible under an inducible promoter. As such, the virus may also be an expression vector from which the polypeptide may be expressed. For example, an engineered virus comprises a suicide gene expressible under an inducible promoter, such as CMV. Methods of cancer treatment involving such engineered virus optionally comprise inducing expression of the suicide gene to kill or otherwise prevent the virus from propagating. Accordingly, the viral infection may be controlled by a physician according to the needs of the patient. In some embodiments, the suicide gene encodes a protein that converts a prodrug into a cytotoxic agent. In some embodiments, the suicide gene encodes thymidine kinase. In some embodiments, the suicide gene encodes cytosine deaminase. In some embodiments, the suicide gene encodes purine nucleoside phosphorylase. In some embodiments, the suicide gene encodes uracil phosphoribosyl transferase. In some embodiments, the suicide gene encodes thymidylate kinase.

In some embodiments, a virus comprises a genetic modification that affects expression of a viral gene. For example, a mutation in a virulence gene that contributes to the pathogenicity of the virus to a host organism such that the expression of that gene is significantly decreased, or wherein the gene product is rendered nonfunctional, or its ability to function is significantly decreased. In some cases, the genetic modification compromises the ability of the virus to replicate, or to replicate in non-cancerous or non-dividing cells.

In some embodiments, the virus is engineered to place at least one viral protein necessary for viral replication under the control of a tumor-specific promoter. In some embodiments, a gene that encodes a cytotoxic agent is placed under the control of a tumor-specific promoter. For example, cytotoxic agents include toxins, prodrugs, cytokines, and chemokines.

As a non-limiting example, a virus comprises a flavivirus or a portion of a flavivirus. In some cases, the flavivirus is within a phylogenetic group of mosquito-borne flaviviruses (MBFV). For example, the MBFV comprises viruses associated with Aedes spp. mosquitoes, and the flavivirus is a virus associated with the Aedes spp. mosquito. Examples of flaviviruses include Zika virus (ZIKV), spondweni virus, and kedougous virus. In some embodiments, a virus comprises a nucleic acid sequence at least about 70%, 75%, 80%, 85%, 90%, or 95% homologous or identical to a virus having a genome of SEQ ID NO: 1. In some cases, a virus encodes for a capsid protein comprising an amino acid sequence at least about 70%, 75%, 80%, 85%, 90%, or 95% homologous or identical to a sequence selected from SEQ ID NO: 2. In some cases, a virus encodes for a membrane protein comprising an amino acid sequence at least about 70%, 75%, 80%, 85%, 90%, or 95% homologous or identical to a sequence selected from SEQ ID NO: 2. In some cases, a virus encodes for an envelope protein comprising an amino acid sequence at least about 70%, 75%, 80%, 85%, 90%, or 95% homologous or identical to a sequence selected from SEQ ID NO: 2. In some cases, a virus encodes for a non-structural protein comprising an amino acid sequence at least about 70%, 75%, 80%, 85%, 90%, or 95% homologous or identical to a sequence selected from SEQ ID NO: 2.

In one aspect, provided herein is a Zika virus and/or an engineered virus derived from a Zika virus, both of which are referred to herein as a Zika virus. Zika viruses may be derived from any laboratory strain or clinical isolate (non-laboratory strain) of Zika virus. A number of Zika viruses are known in the art. In some embodiments, a virus is an engineered virus derived from a Zika virus having a genome having SEQ ID NO: 1. In some cases, the engineered virus has at least about 50% sequence homology and less than about 100% sequence homology to the nucleic acid from which it was derived. The degree of homology or percent identity between a native and a modified or engineered sequence can be determined, for example, by comparing the two sequences using freely available computer programs commonly employed for this purpose on the World Wide Web, such as provided by BLAST by the National Center for Biotechnology Information (NCBI).

Another non-limiting example of a virus is one based on or derived from a virus of the Herpesviridae family, or a portion of Herpesviridae virus. The Herpesviridae family includes the Betaherpesvirinae subfamily, of which cytomegalovirus is a member. Accordingly, a virus useful for the methods and compositions herein is a cytomegalovirus, or a virus derived therefrom. In some embodiments, a virus comprises a nucleic acid sequence at least about 70%, 75%, 80%, 85%, 90%, or 95% homologous or identical to a virus having a genome of a cytomegalovirus. cytomegaloviruses may be derived from any laboratory strain or clinical isolate (non-laboratory strain). A number of cytomegaloviruses are known in the art. In some embodiments, a virus is an engineered virus derived from a cytomegalovirus. In some cases, the engineered virus has at least about 50% sequence homology and less than about 100% sequence homology to the nucleic acid from which it was derived.

In another aspect, provided herein is a nucleic acid comprising the genome of an engineered virus. Another aspect relates to a host cell comprising the virus or a nucleic acid comprising the genome of the virus.

### Methods of Use

Viruses and viral compositions disclosed herein are useful for treating or preventing one or more diseases, conditions, and/or symptoms of cancer. In some cases, the methods promote an immune response to cancer. Exemplary methods comprise contacting a cancerous cell with an effective amount of a virus, such as a Zika virus, and/or an additional agent, such as an immunostimulatory agent. The contacting may be carried out in vitro (e.g., in biochemical and/or cellular assays), in vivo in a non-human animal, and in vivo in mammals, including humans. In some cases, contacting involves bringing a cancer cell and a composition comprising a virus and/or additional agent provided herein into sufficient proximity such that the composition exerts an effect on the cancer cell. Contacting includes physical interaction between the composition and a cancer cell, as well as interactions that do not require physical interaction. Contacting includes administering an effective amount of a composition to a subject comprising the cancer cell such that the composition impairs cancer cell growth, division and/or induces cancer cell death. In some embodiments, contacting by administration includes intravenous administration, intraperitoneal administration, intramuscular administration, intracoronary administration, intraarterial administration, subcutaneous administration, transdermal delivery, intratracheal administration, subcutaneous administration, intraarticular administration, intraventricular administration, inhalation, intracerebral, nasal, oral, pulmonary administration, impregnation of a catheter, and direct injection into a tissue or tumor of the subject.

Subjects include animals, such as humans, other higher primates, lower primates, and animals of veterinary importance, such as dogs, cats, horses, sheep, goats, and cattle and the like. Subjects also include animals for use in studies, for example, mice, rats and other rodents.

Provided is a method for treating cancer in a subject comprising administration of an effective amount of a pharmaceutical composition that includes a virus provided herein and/or at least one immunostimulatory agent to said subject. An effective amount or therapeutically effective amount of a composition is a quantity sufficient to achieve a desired therapeutic and/or prophylactic effect, e.g., an amount which results in the prevention of, or a decrease in, the symptoms associated with a cancer. The amount of a composition administered to the subject may depend on the type and severity of the cancer and on the characteristics of the individual, such as general health, age, sex, body weight and tolerance to drugs. In some embodiments, an effective amount of a virus is administered to a subject having cancer in an amount sufficient to induce oncolysis, the disruption or lysis of a cancer cell, slowing, inhibition and/or reduction in the growth or size of a tumor, and includes the eradication of the tumor in certain instances. In some embodiments, an effective amount of a virus is administered to a subject having cancer in an amount sufficient to attenuate or halt division of a cancer cell.

In some embodiments, the viral composition comprises a heterologous nucleic acid sequence that encodes a suicide gene. In some embodiments, the suicide gene encodes a protein that converts a prodrug administered to the subject into a cytotoxic agent thereby killing the cancer cells. In some embodiments, the suicide gene is thymidine kinase and the prodrug administered to the subject is selected from the group consisting of ganciclovir, ganciclovir elaidic acid ester, penciclovir, acyclovir, valacyclovir, (E)-5-(2-bromovinyl)-2′deoxyuridine, and zidovudine. In some embodiments, the suicide gene is cytosine deaminase and the prodrug administered to the subject 5-fluorocytosine. In some embodiments, the suicide gene is purine nucleoside phosphorylase and the prodrug administered to the subject is 6-methylpurine deoxyriboside or fludarabine. In some embodiments, the suicide gene is uracil phosphoribosyl transferase and the prodrug administered to the subject is 5-fluorocytosine or 5-fluorouracil. In some embodiments, the suicide gene is thymidylate kinase and the prodrug administered to the subject is azidothymidine.

Treatment of cancer includes amelioration, cure, and/or maintenance of a cure (i.e. the prevention or delay of relapse) and/or its associated symptoms. In some cases, treatment comprises eliciting an immune response in a subject to a cancer cell by administering to the subject a composition comprising a virus and/or immunostimulatory agent described herein. For example, a subject is successfully treated for a cancer if after receiving a therapeutic amount of the composition described herein, the subject shows observable and/or measurable reduction in or absence of one or more signs and symptoms of the cancer, e.g., reduction in the number of cancer cells or absence of the cancer cells; reduction in the tumor size; inhibition (i.e., slow to some extent and preferably stop) of tumor metastasis; inhibition, to some extent, of tumor growth; increase in length of remission, and/or relief to some extent, of one or more of the symptoms associated with the specific cancer; reduced morbidity and mortality, and improvement in quality of life issues. Treatment also includes preventing the cancer from becoming worse, slowing the rate of progression, and/or preventing the cancer from re-occurrence after initial elimination. A suitable dose and therapeutic regimen may vary depending upon the specific virus used, the mode of delivery of the virus, and whether it is used alone or in combination with one or more other viruses or immunostimulatory agent.

In some embodiments, a method comprises administration of a virus and/or immunostimulatory agent provided herein to a subject having a tumor, such that tumor cell growth or proliferation is inhibited. In some cases, tumor growth or proliferation is reduced by at least about 10%, 20%, 30%, 40%, 50%, 60%, 70%, 80%, 90% 95% or 100%, and includes inhibition of tumor cell division and/or induction of tumor cell death.

In some embodiments, a method comprises administration of a virus and/or immunostimulatory agent provided herein to a subject having a tumor, such that tumor cell progression (e.g., tumorigenesis, tumor growth and proliferation, invasion and metastasis) is inhibited. In some cases, inhibiting tumor progression refers to inhibiting the development, growth, proliferation, or spreading of a tumor, including without limitation the following effects: inhibition of growth of cells in a tumor, (2) inhibition, to some extent, of tumor growth, including slowing down or complete growth arrest; (3) reduction in the number of tumor cells; (4) reduction in tumor size; (5) inhibition (i.e., reduction, slowing down or complete stopping) of tumor cell infiltration into adjacent peripheral organs and/or tissues; (6) inhibition (i.e. reduction, slowing down or complete stopping) of metastasis; (7) increase in the length of survival of a patient or patient population following treatment for a tumor; and/or (8) decreased mortality of a patient or patient population at a given time point following treatment for a tumor. In some cases, tumor progression is reduced by at least about 10%, 20%, 30%, 40%, 50%, 60%, 70%, 80%, 90%, 95% or 100%.

In some embodiments, a therapeutic benefit is achieved after administration of a composition herein. A therapeutic benefit includes anything that promotes or enhances the well-being of the subject with respect to the medical treatment of his/her condition, which includes treatment of pre-cancer, cancer, and hyperproliferative diseases. For example, extension of the subject's life by any period of time, decrease or delay in the neoplastic development of the disease, decrease in hyperproliferation, reduction in tumor growth, delay of metastases, reduction in cancer cell or tumor cell proliferation rate, and a decrease in pain to the subject that can be attributed to the subject's condition.

Administration of a pharmaceutical composition to a subject is by means which the virus and/or immunostimulatory agent contained therein will contact a target cell. The specific route will depend upon certain variables such as the cancer cell, and can be determined by the skilled practitioner. Suitable methods of administering a composition comprising a pharmaceutical composition of the present invention to a patient include any route of in vivo administration that is suitable for delivering a virus and/or immunostimulatory agent to a patient. Exemplary methods of in vivo administration include, but are not limited to, intravenous administration, intraperitoneal administration, intramuscular administration, intracoronary administration, intraarterial administration (e.g., into a carotid artery), subcutaneous administration, transdermal delivery, intratracheal administration, subcutaneous administration, intraarticular administration, intraventricular administration, inhalation (e.g., aerosol), intracerebral, nasal, oral, pulmonary administration, impregnation of a catheter, and direct injection into a tissue. In an embodiment where the target cells are in or near a tumor, a preferred route of administration is by direct injection into the tumor or tissue surrounding the tumor.

Various routes of administration are contemplated for various tumor types. Where discrete tumor mass, or solid tumor, may be identified, a variety of direct, local and regional approaches may be taken. For example, the tumor is directly injected with the composition. A tumor bed may be treated prior to, during or after resection and/or other treatment(s). Following resection or other treatment(s), one generally will deliver the adenovirus by a catheter having access to the tumor or the residual tumor site following surgery. Methods of treating cancer include treatment of a tumor as well as treatment of the region near or around the tumor. This includes body cavities in which the tumor lies, as well as cells and tissue that are next to the tumor.

### Combinations

In some embodiments, a pharmaceutical composition comprising a virus is administered in combination with another agent. In some cases the other agent is an immunostimulatory agent. Non-limiting examples of immunostimulatory agents include a Toll-like receptor (TLR) agonist, granulocyte-macrophage colony-stimulating factor (GMCSF), heat shock protein (Hsp) 70, Hsp90, Hsp110, CpG oligonucleotide, and combinations thereof. Exemplary TLR agonists include, but are not limited to, lipopolysaccharide (LPS), imiquimod (TLR7 agonist), rintatolimod, and resiquimod. In some cases, an immunostimulatory agent is an adjuvant. Non-limiting examples of adjuvants include an aluminum salt (e.g., aluminum phosphate, aluminum hydroxide, aluminum potassium sulfate), an oil-in-water emulsion (e.g., squalene, polysorbate 80 (Tween 80), sorbitan trioleate, alpha-tocopherol, MF59, Adjuvant System 03 (AS03)), liposome, glucopyranosyl lipid adjuvant formulated in a stable emulsion (GLA-SE), synthetic oligodeoxynucleotide (ODN1a), a peptide comprising KLKL(5)KLK (SEQ ID NO: 3), IC31CD, dimethyldioctadecyl-ammonium (DDA), trehalose 6,6-dibehenate (TDB), CAF01, monophosphoryl lipid A, and TLR agonist.

In some embodiments, an immunostimulatory agent is an immunotherapeutic. In some cases, the immunotherapeutic is a tumor-specific monoclonal antibody that targets cancer-specific antigens of the cancer cell. In some cases, the immunotherapeutic is a cytokine, such as interferon-alpha (IFN-α), interleukin (IL)-2, and IL-12. In some cases, the immunotherapeutic is part of an adoptive cell transfer (ACT) approach that includes, but is not limited to, a tumor infiltrating lymphocytes (TIL), cytokine-induced killer (CIK) cells, and cascade-primed (CAPRI) cells. In some cases, the immunotherapeutic is a CAR T-cell. In some cases, the immunotherapeutic is an immune checkpoint inhibitor. Non-limiting examples of immune checkpoint inhibitors include an anti-CTLA-4 antibody and antigen binding fragment thereof, an anti-PD-1 antibody and an antigen binding fragment thereof, and an anti-PD-1L antibody and an antigen binding fragment thereof. In some cases, the anti-CTLA-4 antibody is ipilimumab. In some cases, the anti-PD-1 antibody is pembrolizumab, nivolumab, BGB-A317, or a combination thereof. In some cases, the anti-PD-1L antibody is avelumab, atezolizumab, durvalumab, or a combination thereof.

In some cases, a virus is administered with an agent effective in the treatment of cancer (“anti-cancer agent”). An anti-cancer agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. Anti-cancer agents include biological agents, chemotherapy agents, and radiotherapy agents. The pharmaceutical composition provided herein and the anti-cancer agent may be administered simultaneously, e.g. as a combined preparation or as separate preparations one administered immediately after the other. Alternatively, they may be administered separately and sequentially, where one agent is administered and then the other administered later after a predetermined time interval. A pharmaceutical composition provided herein and an anti-cancer agent may be administered simultaneously, separately, or sequentially which may allow the two agents to be present in the tumor requiring treatment at the same time and thereby provide a combined therapeutic effect, which may be additive or synergistic.

It is contemplated that the pharmaceutical composition is useful in conjunction with surgery and/or chemotherapeutic, radiotherapeutic, immunotherapeutic or other biological intervention. Methods may comprise administering to the patient a second therapy, wherein the second therapy is anti-angiogenic therapy, chemotherapy, immunotherapy, surgery, radiotherapy, immunosuppresive agents, or gene therapy with a therapeutic polynucleotide. In some cases, the second therapy is administered to the patient before, at the same time, or after administration of the pharmaceutical composition provided herein.

### Cancer

As used herein, cancer is a class of diseases characterized by uncontrolled cellular growth. Cancer includes all types of hyperproliferative growth, hyperplastic growth, neoplastic growth, cancerous growths or oncogenic processes, metastatic tissues or malignantly transformed cells, tissues, or organs, irrespective of histopathologic type or stage of invasiveness.

In some embodiments, a composition provided herein (virus and/or immunostimulatory agent), prevents and/or attenuates tumor cell growth when administered to a patient having cancer. In some embodiments, a composition herein prevents and/or attenuates tumor cell invasion when administered to a patient having cancer. In some embodiments, a composition herein prevents and/or attenuates tumor cell metastasis when administered to a patient having cancer. In some embodiments, a composition herein promotes and immune response to a cancerous cell when administered to a patient having the cancer cell. In some embodiments, a composition herein a virus comprising or derived from a flavivirus, such as a Zika virus. In some embodiments, a composition herein a virus comprising or derived from a Herpesviridae virus, such as a cytomegalovirus. In some embodiments, a composition herein comprises an immunostimulatory agent.

Types of cancer include, but are not limited to, solid tumors (such as those of the bladder, bowel, brain, breast, endometrium, heart, kidney, lung, liver, uterus, lymphatic tissue (lymphoma), ovary, pancreas or other endocrine organ (thyroid), prostate, skin (melanoma or basal cell cancer)) and hematological tumors (such as the leukemias and lymphomas) at any stage of the disease, with or without metastases. In some embodiments, the cancer comprises a brain tumor. Non-limiting examples of tumors treatable with a composition provided herein include adenoma, angio-sarcoma, astrocytoma, epithelial carcinoma, germinoma, glioblastoma, glioma, hamartoma, hemangioendothelioma, hemangiosarcoma, hematoma, hepato-blastoma, leukemia, lymphoma, medulloblastoma, melanoma, neuroblastoma, osteosarcoma, retinoblastoma, rhabdomyosarcoma, sarcoma, and teratoma. Glioma refers to a tumor originating in the neuroglia of the brain or spinal cord. Gliomas are derived from the glial cell types such as astrocytes and oligodendrocytes, thus gliomas include astrocytomas and oligodendrogliomas, as well as anaplastic gliomas, glioblastomas, and ependymomas. Additional brain tumors include meningiomas, ependymomas, pineal region tumors, choroid plexus tumors, neuroepithelial tumors, embryonal tumors, peripheral neuroblastic tumors, tumors of cranial nerves, tumors of the hemopoietic system, germ cell tumors, and tumors of the sellar region.

### Dosing and Treatment Regimens

Administration frequencies for a pharmaceutical composition comprising a virus and/or immunostimulatory agent provided herein may vary based on the method being practiced, the physical characteristics of the subject, the severity of the cancer, cancer type, and the formulation and the means used to administer the composition. Non-limiting exemplary administration frequencies include 6, 5, 4, 3, 2 or once daily, every other day, every third day, every fourth day, every fifth day, every sixth day, once weekly, every eight days, every nine days, every ten days, bi-weekly, monthly, bi-monthly, every three months, every four months, every five months, and every six months. In certain aspects, the pharmaceutical composition is administered once daily. In some embodiments, the pharmaceutical composition is administered continuously.

The duration of treatment will be based on the condition being treated and may be determined by the attending physician. The duration of administration, in many instances, varies depending on a number of factors. Exemplary factors include, without limitation, patient response, severity of symptoms, and cancer type. Under some conditions, treatment is continued for a number of days, weeks, or months. Under other conditions, complete treatment is achieve through administering one, two or three dose of the pharmaceutical composition over the entire course of treatment. In certain aspects, complete treatment can be achieved using a single dose of the pharmaceutical composition.

In certain embodiments wherein the patient's condition does not improve, upon the doctor's discretion the administration of the virus and/or immunostimulatory agent is administered chronically, that is, for an extended period of time, including throughout the duration of the patient's life in order to ameliorate or otherwise control or limit the symptoms of the patient's disease or condition.

In certain embodiments wherein a patient's status does improve, the dose of composition being administered may be temporarily reduced or temporarily suspended for a certain length of time (i.e. a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including 10%, 15%, 20%, 25%, 30%, 35%, 40%, 45%, 50%, 55%, 60%, 65%, 70%, 75%, 80%, 85%, 90%, 95%, and 100%.

In certain embodiments the dose of the composition being administered is temporarily reduced or temporarily suspended for a certain length of time (i.e. a “drug diversion”). In specific embodiments, the length of the drug diversion is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug diversion is, by way of example only, by 10%-100%, including 10%, 15%, 20%, 25%, 30%, 35%, 40%, 45%, 50%, 55%, 60%, 65%, 70%, 75%, 80%, 85%, 90%, 95%, and 100%. After a suitable length of time, the normal dosing schedule is optionally reinstated.

In some embodiments, as a patient is started on a regimen of a virus and/or immunostimulatory agent, the patient is also weaned off (e.g., step-wise decrease in dose) a second treatment regimen.

In some embodiments, once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of a given composition varies depending upon factors such as the particular virus and/or immunostimulatory agent, disease condition and its severity, the identity (e.g., weight, sex) of the subject in need of treatment, but can nevertheless be determined according to the particular circumstances surrounding the case. In some cases, doses employed for adult human treatment are typically in the range of 0.01 mg-5000 mg per day. In some cases, doses employed for adult human treatment are from about 1 mg to about 1000 mg per day. In some embodiments, the desired dose is conveniently presented in a single dose or in divided doses administered simultaneously (or over a short period of time) or at appropriate intervals, for example as two, three, four or more sub-doses per day.

In some embodiments, the daily dosages appropriate for a immunostimulatory agent described herein are from about 0.01 to about 10 mg/kg per body weight. In some cases, an indicated daily dosage in a large mammal, including, but not limited to, humans, is in the range from about 0.5 mg to about 1000 mg, optionally administered in divided doses, including, but not limited to, up to four times a day. In some embodiments, the daily dosage is administered in extended release form. In certain embodiments, suitable unit dosage forms for oral administration comprise from about 1 to 500 mg immunostimulatory agent. In other embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the immunostimulatory agent used, the disease or condition to be treated, the mode of administration, the requirements of the individual subject, the severity of the disease or condition being treated, and the judgment of the practitioner.

In some embodiments, an immunostimulatory agent is administered an amount from about 25 mg to about 100 mg per dose. In some embodiments, an immunostimulatory agent is administered an amount from about 100 mg to about 200 mg per dose. In some embodiments, an immunostimulatory agent is administered an amount from about 200 mg to about 400 mg per dose. In some embodiments, an immunostimulatory agent is administered an amount from about 400 mg to about 500 mg. In some embodiments, an immunostimulatory agent is administered an amount from about 500 mg to about 1,500 mg.

In some embodiments, administration of a virus depending on the kind of virus and titer, and includes delivery of 1 to 100, 10 to 50, 100-1000, or up to 1*104, 1*105, 1*106, 1*107, 1*108, 1*109, 1*1010, 1*1011, 1*1012 or 1*1013 infectious particles to the patient in a pharmaceutically acceptable composition.

### Pharmaceutical Compositions and Formulations

Provided herein are viruses and viral compositions comprising viruses and another agent formulated into pharmaceutical compositions. As a non-limiting example, the other agent is an immunostimulatory agent. In some cases, the other agent is an adjuvant. Pharmaceutical compositions are formulated in a conventional manner using one or more pharmaceutically acceptable inactive ingredients that facilitate processing of the active agent into preparations that can be used pharmaceutically. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999), herein incorporated by reference for such disclosure. For some virus delivery methods, a pharmaceutically acceptable vehicle is selected from known pharmaceutically acceptable vehicles for delivery, and should be one in which the virus is stable.

Further provided herein are pharmaceutical compositions that include a virus and/or another agent; a pharmaceutically acceptable inactive ingredient; other medicinal or pharmaceutical agent; carrier; adjuvant; preserving, stabilizing, wetting or emulsifying agent; solution promoter; salt; buffer; excipients; binder; filling agent; suspending agent; flavoring agent; sweetening agents; disintegrating agent; dispersing agent; surfactants; lubricant; colorant; diluent; solubilizer; moistening agent; plasticizers; penetration enhancer; anti-foam agent; antioxidant; preservative; or a combination thereof.

A composition as described herein, in various embodiments, comprises two or more pharmaceutical compositions. In some embodiments, a pharmaceutical composition comprises one or more viruses, for example, comprising or derived from a flavivirus, such as Zika virus. In some embodiments, a pharmaceutical composition comprises one or more immunostimulatory agents, or a pharmaceutically acceptable salt or solvate thereof. The two or more pharmaceutical compositions are administered together or separately.

In some embodiments, a pharmaceutical composition comprises at least one agent as an active agent in free-acid or free-base form, or in a pharmaceutically acceptable salt form.

In some embodiments, pharmaceutically acceptable salts are obtained by reacting an agent described herein with an acid to provide a “pharmaceutically acceptable acid addition salt.” In some embodiments, the agent described herein (i.e. free base form) is basic and is reacted with an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; monomethyl fumarate, naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, pharmaceutically acceptable salts are obtained by reacting an agent described herein with a base to provide a “pharmaceutically acceptable base addition salt.”

In some embodiments, the agent described herein is acidic and is reacted with a base. In such situations, an acidic proton of the agent described herein is replaced by a metal ion, e.g., lithium, sodium, potassium, magnesium, calcium, or an aluminum ion. In some cases, agents described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, agents described herein form salts with amino acids such as, but not limited to, arginine, lysine, and the like. Acceptable inorganic bases used to form salts with agents that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the agents provided herein are prepared as a sodium salt, calcium salt, potassium salt, magnesium salt, meglumine salt, N-methylglucamine salt or ammonium salt.

It should be understood that a reference to a pharmaceutically acceptable salt includes the solvent addition forms. In some embodiments, solvates contain either stoichiometric or non-stoichiometric amounts of a solvent, and are formed during the process of isolating or purifying the agent with pharmaceutically acceptable solvents such as water, ethanol, and the like. Hydrates are formed when the solvent is water, or alcoholates are formed when the solvent is alcohol. Solvates of agents described herein are conveniently prepared or formed during the processes described herein. In addition, the agents provided herein optionally exist in unsolvated as well as solvated forms. The solvated forms of the agents are also considered to be disclosed herein.

In addition, the methods and pharmaceutical compositions described herein include the use of N-oxides (if appropriate), crystalline forms, amorphous phases, as well as active metabolites of these agents having the same type of activity.

In some embodiments, the agents exist as tautomers. All tautomers are included within the scope of the agents presented herein. As such, it is to be understood that an agent or a salt thereof may exhibit the phenomenon of tautomerism whereby two chemical agents that are capable of facile interconversion by exchanging a hydrogen atom between two atoms, to either of which it forms a covalent bond. Since the tautomeric agents exist in mobile equilibrium with each other they may be regarded as different isomeric forms of the same agent. It is to be understood that the formulae drawings within this specification can represent only one of the possible tautomeric forms. However, it is also to be understood that the present disclosure encompasses any tautomeric form, and is not to be limited merely to any one tautomeric form utilized within the formulae drawings. The formulae drawings within this specification can represent only one of the possible tautomeric forms and it is to be understood that the specification encompasses all possible tautomeric forms of the agents drawn not just those forms which it has been convenient to show graphically herein.

In some embodiments, an agent exist as enantiomers, diastereomers, or other steroisomeric forms. The agents disclosed herein include all enantiomeric, diastereomeric, and epimeric forms as well as mixtures thereof.

In some embodiments, agents described herein are prepared as prodrugs. A prodrug refers to an agent that is converted into the parent drug in vivo. Prodrugs are often useful because in some situations they are easier to administer than the parent drug. For example, in some cases a prodrug is bioavailable by oral administration whereas the parent is not. In some cases, the prodrug has improved solubility in pharmaceutical compositions over the parent drug. In a non-limiting example, a prodrug of an agent described herein is administered as an ester (the prodrug) to facilitate transmittal across a cell membrane where water solubility is detrimental to mobility but which then is metabolically hydrolyzed to the carboxylic acid, the active entity, once inside the cell where water-solubility is beneficial. A further example of a prodrug is a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the agent. In certain embodiments, a prodrug is enzymatically metabolized by one or more steps or processes to the biologically, pharmaceutically or therapeutically active form of the agent. Prodrugs metabolized in vivo to produce an agent as set forth herein are included within the scope of the claims. In some cases, an agent described herein is a prodrug for another derivative or active compound.

In certain embodiments, compositions provided herein include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, formulations described herein benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

The pharmaceutical compositions described herein, which include an agent are formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

In one aspect, an agent is formulated into a pharmaceutical composition suitable for intramuscular, subcutaneous, or intravenous injection. In some embodiments, formulations suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. In some cases it is desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections or drips or infusions, agents described herein are formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations include aqueous or nonaqueous solutions, preferably with physiologically compatible buffers or excipients.

Parenteral injections may involve bolus injection or continuous infusion. Formulations for injection may be presented in unit dosage form, e.g., in ampoules or in multi-dose containers, with an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

For administration by inhalation, an agent is formulated for use as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the agent described herein and a suitable powder base such as lactose or starch.

Representative intranasal formulations are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. The choice of suitable carriers is dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. Preferably, the nasal dosage form should be isotonic with nasal secretions.

Buccal formulations that include an agent are administered using a variety of formulations known in the art. In addition, the buccal dosage forms described herein can further include a bioerodible (hydrolysable) polymeric carrier that also serves to adhere the dosage form to the buccal mucosa. For buccal or sublingual administration, the compositions may take the form of tablets, lozenges, or gels formulated in a conventional manner.

For intravenous injections, a composition is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients. Parenteral injections optionally involve bolus injection or continuous infusion. Formulations for injection are optionally presented in unit dosage form, e.g., in ampoules or in multi dose containers, with an added preservative. In some embodiments, a pharmaceutical composition described herein is in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of an agent that modulates the activity of a carotid body in water soluble form. Additionally, suspensions of an agent that modulates the activity of a carotid body are optionally prepared as appropriate, e.g., oily injection suspensions.

Pharmaceutical preparations for oral use are obtained by mixing one or more solid excipient with one or more of the agents described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active compound doses.

In some embodiments, pharmaceutical formulations of an agent are in the form of a capsules, including push-fit capsules made of gelatin, as well as soft, sealed capsules made of gelatin and a plasticizer, such as glycerol or sorbitol. The push-fit capsules contain the active ingredients in admixture with filler such as lactose, binders such as starches, and/or lubricants such as talc or magnesium stearate and, optionally, stabilizers. In soft capsules, the agents are dissolved or suspended in suitable liquids, such as fatty oils, liquid paraffin, or liquid polyethylene glycols. In some embodiments, stabilizers are added. A capsule may be prepared, for example, by placing the bulk blend of the formulation of the agent described above, inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule is swallowed whole or the capsule is opened and the contents sprinkled on food prior to eating.

In some embodiments, a solid oral dosage forms are prepared by mixing an agent with one or more of the following: antioxidants, flavoring agents, and carrier materials such as binders, suspending agents, disintegration agents, filling agents, surfactants, solubilizers, stabilizers, lubricants, wetting agents, and diluents. In some cases, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder, a capsule, solid dispersion, solid solution, bioerodible dosage form, controlled release formulations, pulsatile release dosage forms, multiparticulate dosage forms, beads, pellets, granules. In other embodiments, the pharmaceutical formulation is in the form of a powder. Compressed tablets are solid dosage forms prepared by compacting the bulk blend of the formulations described above. In various embodiments, tablets will include one or more flavoring agents. In some embodiments, the tablets will include a film surrounding the final compressed tablet. In some embodiments, the film coating can provide a delayed release of the agent from the formulation. In some embodiments, the film coating aids in patient compliance (e.g., Opadry® coatings or sugar coating). Film coatings including Opadry® typically range from about 1% to about 3% of the tablet weight. In some embodiments, solid dosage forms, e.g., tablets, effervescent tablets, and capsules, are prepared by mixing particles of an agentwith one or more pharmaceutical excipients to form a bulk blend composition. The bulk blend is readily subdivided into equally effective unit dosage forms, such as tablets, pills, and capsules. In some embodiments, the individual unit dosages include film coatings. These formulations are manufactured by conventional formulation techniques.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

Suitable disintegrants for use in the solid dosage forms described herein include, but are not limited to, natural starch such as corn starch or potato starch, a pregelatinized starch, or sodium starch glycolate, a cellulose such as methylcrystalline cellulose, methylcellulose, microcrystalline cellulose, croscarmellose, or a cross-linked cellulose, such as cross-linked sodium carboxymethylcellulose, cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a gum such as agar, guar, locust bean, Karaya, pectin, or tragacanth, sodium starch glycolate, bentonite, sodium lauryl sulfate, sodium lauryl sulfate in combination starch, and the like.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose, hydroxypropylmethylcellulose, hydroxypropylmethylcellulose acetate stearate, hydroxyethylcellulose, hydroxypropylcellulose, ethylcellulose, and microcrystalline cellulose, microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose, glucose, dextrose, molasses, mannitol, sorbitol, xylitol, lactose, a natural or synthetic gum such as acacia, tragacanth, ghatti gum, mucilage of isapol husks, starch, polyvinylpyrrolidone, larch arabogalactan, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. Binder levels of up to 70% in tablet formulations is common.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™ PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives should be taken as merely exemplary, and not limiting, of the types of additives that can be included in solid dosage forms of the pharmaceutical compositions described herein. The amounts of such additives can be readily determined by one skilled in the art, according to the particular properties desired.

In some embodiments, the solid dosage forms described herein are formulated as enteric coated delayed release oral dosage forms, i.e., as an oral dosage form of a pharmaceutical composition as described herein which utilizes an enteric coating to affect release in the small intestine or large intestine. In one aspect, the enteric coated dosage form is a compressed or molded or extruded tablet/mold (coated or uncoated) containing granules, powder, pellets, beads or particles of the active ingredient and/or other composition components, which are themselves coated or uncoated. In one aspect, the enteric coated oral dosage form is in the form of a capsule containing pellets, beads or granules, which include an immunostimulatory agent, that are coated or uncoated. Any coatings should be applied to a sufficient thickness such that the entire coating does not dissolve in the gastrointestinal fluids at pH below about 5, but does dissolve at pH about 5 and above. Coatings include shellac, which dissolves in media of pH>7; acrylic polymers such as methacrylic acid copolymers and ammonium methacrylate copolymers; Eudragit series E, L, S, RL, RS and NE (Rohm Pharma) available as solubilized in organic solvent, aqueous dispersion, or dry powders; and Poly Vinyl Acetate Phthalate (PVAP) which dissolves in pH>5, and it is less permeable to water vapor and gastric fluids. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine. Conventional coating techniques such as spray or pan coating are employed to apply coatings. The coating thickness must be sufficient to ensure that the oral dosage form remains intact until the desired site of topical delivery in the intestinal tract is reached.

In various embodiments, the particles of an agent and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In other embodiments, a powder including an agent is formulated to include one or more pharmaceutical excipients and flavors. Such a powder is prepared, for example, by mixing the agent and optional pharmaceutical excipients to form a bulk blend composition. Additional embodiments also include a suspending agent and/or a wetting agent. This bulk blend is uniformly subdivided into unit dosage packaging or multi-dosage packaging units.

In still other embodiments, effervescent powders are also prepared. Effervescent salts have been used to disperse medicines in water for oral administration.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents. Exemplary useful microencapsulation materials include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG,HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

Liquid formulation dosage forms for oral administration are optionally aqueous suspensions selected from the group including, but not limited to, pharmaceutically acceptable aqueous oral dispersions, emulsions, solutions, elixirs, gels, and syrups. In addition to a virus and/or agent the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further includes a crystal-forming inhibitor.

In some embodiments, the pharmaceutical formulations described herein are self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients.

In some embodiments, pharmaceutical dosage forms are formulated to provide a controlled release of a virus and/or another agent. Controlled release refers to the release of the virus and/or another agent from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In other embodiments, the formulations described herein are delivered using a pulsatile dosage form. A pulsatile dosage form is capable of providing one or more immediate release pulses at predetermined time points after a controlled lag time or at specific sites. In some embodiments, the pulsatile dosage form includes at least two groups of particles, (i.e. multiparticulate) each containing the formulation described herein. The first group of particles provides a substantially immediate dose of the agent upon ingestion by a mammal. The first group of particles can be either uncoated or include a coating and/or sealant. In one aspect, the second group of particles comprises coated particles. The coating on the second group of particles provides a delay of from about 2 hours to about 7 hours following ingestion before release of the second dose. Suitable coatings for pharmaceutical compositions are described herein or known in the art.

In some embodiments, pharmaceutical formulations are provided that include particles of an agent and at least one dispersing agent or suspending agent for oral administration to a subject. The formulations may be a powder and/or granules for suspension, and upon admixture with water, a substantially uniform suspension is obtained.

In some embodiments, liquid formulations are provided having inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

In some embodiments, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, pharmaceutical compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In one embodiment, the aqueous suspensions and dispersions described herein remain in a homogenous state, as defined in The USP Pharmacists' Pharmacopeia (2005 edition, chapter 905), for at least 4 hours. In one embodiment, an aqueous suspension is re-suspended into a homogenous suspension by physical agitation lasting less than 1 minute. In still another embodiment, no agitation is necessary to maintain a homogeneous aqueous dispersion. Examples of disintegrating agents for use in the aqueous suspensions and dispersions include, but are not limited to, a starch, e.g., a natural starch such as corn starch or potato starch, a pregelatinized starch, or sodium starch glycolate; a cellulose such as methylcrystalline cellulose, methylcellulose, croscarmellose, or a cross-linked cellulose, such as cross-linked sodium carboxymethylcellulose, cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein include, for example, hydrophilic polymers, electrolytes, Tween ° 60 or 80, PEG, polyvinylpyrrolidone, and the carbohydrate-based dispersing agents such as, for example, hydroxypropylcellulose and hydroxypropyl cellulose ethers, hydroxypropyl methylcellulose and hydroxypropyl methylcellulose ethers, carboxymethylcellulose sodium, methylcellulose, hydroxyethylcellulose, hydroxypropylmethyl-cellulose phthalate, hydroxypropylmethyl-cellulose acetate stearate, noncrystalline cellulose, magnesium aluminum silicate, triethanolamine, polyvinyl alcohol (PVA), polyvinylpyrrolidone/vinyl acetate copolymer, 4-(1,1,3,3-tetramethylbutyl)-phenol polymer with ethylene oxide and formaldehyde (also known as tyloxapol), poloxamers; and poloxamines. In other embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl cellulose ethers; hydroxypropyl methylcellulose and hydroxypropyl methylcellulose ethers; carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers; or poloxamines.

Wetting agents suitable for the aqueous suspensions and dispersions described herein include, but are not limited to, cetyl alcohol, glycerol monostearate, polyoxyethylene sorbitan fatty acid esters (e.g., the commercially available Tweens® such as e.g., Tween 20® and Tween 80®, and polyethylene glycols, oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like.

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Suitable viscosity enhancing agents for the aqueous suspensions or dispersions described herein include, but are not limited to, methyl cellulose, xanthan gum, carboxymethyl cellulose, hydroxypropyl cellulose, hydroxypropylmethyl cellulose, Plasdon® S-630, carbomer, polyvinyl alcohol, alginates, acacia, chitosans and combinations thereof. The concentration of the viscosity enhancing agent will depend upon the agent selected and the viscosity desired.

Examples of sweetening agents suitable for the aqueous suspensions or dispersions described herein include, for example, acacia syrup, acesulfame K, alitame, aspartame, chocolate, cinnamon, citrus, cocoa, cyclamate, dextrose, fructose, ginger, glycyrrhetinate, glycyrrhiza (licorice) syrup, monoammonium glyrrhizinate (MagnaSweet®), malitol, mannitol, menthol, neohesperidine DC, neotame, Prosweet® Powder, saccharin, sorbitol, stevia, sucralose, sucrose, sodium saccharin, saccharin, aspartame, acesulfame potassium, mannitol, sucralose, tagatose, thaumatin, vanilla, xylitol, or any combination thereof.

### Kits

In one aspect of the disclosure, provided herein are kits which include one or more reagents or devices for the performance of the methods disclosed herein. In some embodiments, the kit comprises a composition as described herein. In some embodiments, the kit comprises a means to administrate the composition. In some embodiments, one or more of the compositions of a kit comprises a virus and/or one or more other agents, such as an immunostimulatory agent.

In some embodiments, the kit comprises suitable instructions in order to perform the methods of the kit. The instructions may provide information of performing any of the methods disclosed herein, whether or not the methods may be performed using only the reagents provided in the kit. The kit and instructions may require additional reagents or systems.

For use in the therapeutic applications described herein, kits and articles of manufacture are also described herein. In some embodiments, such kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. The containers can be formed from a variety of materials such as glass or plastic. The articles of manufacture provided herein contain packaging materials. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. The container(s) optionally have a sterile access port (for example the container is an intravenous solution bag or a vial having a stopper pierceable by a hypodermic injection needle). Such kits optionally comprise a composition with an identifying description or label or instructions relating to its use in the methods described herein.

A kit will typically include one or more additional containers, each with one or more of various materials (such as reagents, optionally in concentrated form, and/or devices) desirable from a commercial and user standpoint for use of an immunostimulatory agent described herein. Non-limiting examples of such materials include, but not limited to, buffers, diluents, filters, needles, syringes, carrier, package, container, vial and/or tube labels listing contents and/or instructions for use, and package inserts with instructions for use. A set of instructions will also typically be included.

In some embodiments, a label is on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

In certain embodiments, a pharmaceutical composition comprising a virus and/or other agent described herein and optional additional active agent is presented in a pack or dispenser device which can contain one or more unit dosage forms. The pack can for example contain metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions containing an agentprovided herein formulated in a compatible pharmaceutical carrier can also be prepared, placed in an appropriate container, and labeled for treatment of an indicated condition.

### EXAMPLES

### Example 1: Tropism of Zika Virus

A population of neuroepithelial stem (NES) cells derived from human neocortical (NCX) and spinal cord neuroepithelial stem (NES) cells served as an in vitro model for ZIKV-related neuropathogenesis. Postmitotic neurons and NES cells were infected with Zika virus. The ability of Zika virus to infect postmitotic neurons was extremely low (˜3.3%) in comparison to its ability to infect self-renewing NES cells (˜90%).

### Example 2: Cell Culture Assay

U87 human glioma cell line is obtained from American Type Culture Collection and maintained as recommended. Cells are incubated in a multi-well plate and infected with Zika virus at selected values of multiplicity of infection (MOI) in medium. The percentage of mitotic cells and viable cells is assessed.

### Example 3: Mouse Model

Studies are performed in U-87 MG-based intracranial xenografts in nude mice. Treatments comprise intratumoral administration of Zika virus, and a Zika virus and immunostimulatory agent combination. An exemplary dose of Zika virus is 10-1000 viral particles.

Primary studies are focused on survival. At death (cancer-induced or sacrificed), brain tissue is extracted. Tumors are examined using H&E staining and immunohistochemistry for viral proteins and angiogenesis.

### Example 4: Clinical Trail

A Phase 1, dose-escalating, two-part study of Zika virus for glioma is initiated. Primary objectives of the study are to determine the safety, tolerability, feasibility, and biological effect of injecting Zika virus into human brain tumors. To be eligible for the study, patients are required to have histologically-proven, malignant glioma.

Group A is treated with direct intratumoral injection of Zika virus into an area of biopsy-confirmed glioma, and Group B is treated with injection of a divided dose of virus into the resection bed prior to and following glioma excision. The starting dose for both study groups is between 10 and 1000 viral particles, with a plan to dose escalate in half-log increments.

Patients are observed for 28 days following virus injection, and then follow-ups are scheduled to occur at monthly intervals for 4 months, every 2 months for 2 years, and every 4 months for life thereafter for both treatment groups. Patients are monitored for toxicity and symptoms, and evaluated using magnetic resonance imaging (MM), spinal tap, and other tests as appropriate based on clinical standards of care for the duration of the study.

### Example 5: Preparation of Engineered Virus

The viral genome of Zika virus is assembled into Vero cells after electroporation of overlapping synthetic fragments encoding different parts of the viral genome. Briefly, the cDNA sequences of the fragments are cloned into a plasmid and amplified by PCR using primers designed so that adjacent fragments to be assembled share sequence homology at their connecting ends. The first fragment comprises a cytomegalovirus (CMV) promoter upstream of the viral genome to allow for transcription initiation. Infectious virus is recovered by transfection of the assembled cDNA into Vero cells. The resultant viruses are plaque-purified.

For viruses having a heterologous nucleic acid sequence, this sequence is fused into or adjoining a region encoding for a structural protein of the virus in a viral fragment, for example, downstream or within the sequence encoding for the capsid protein. An exemplary heterologous nucleic acid is one encoding for a suicide gene.

TABLE 1
Sequences

| SEQ | | |
|---|---|---|
| ID | | |
| NO | Title | Sequence |
| 1 | Zika virus | AGTTGTTGATCTGTGTGAATCAGACTGCGACAGTTCGAGTTTGAAGCGA |
| | isolate | AAGCTAGCAACAGTATCAACA |
| | ZIKV/H. | GGTTTTATTTTGGATTTGGAAACGAGAGTTTCTGGTCATGAAAAACCCA |
| | sapiens/ | AAAAAGAAATCCGGAGGATTC |
| | Brazil/ | CGGATTGTCAATATGCTAAAACGCGGAGTAGCCCGTGTGAGCCCCTTTG |
| | PE243/2015, | GGGGCTTGAAGAGGCTGCCAG |
| | complete | CCGGACTTCTGCTGGGTCATGGGCCCATCAGGATGGTCTTGGCAATTCT |
| | genome | AGCCTTTTTGAGATTCACGGC |
| | | AATCAAGCCATCACTGGGTCTCATCAATAGATGGGGTTCAGTGGGGAAA |
| | | AAAGAGGCTATGGAAATAATA |
| | | AAGAAGTTCAAGAAAGATCTGGCTGCCATGCTGAGAATAATCAATGCTA |
| | | GGAAGGAGAAGAAGAGACGGG |
| | | GCGCAGATACTAGTGTCGGAATTGTTGGCCTCCTGCTGACCACAGCTAT |
| | | GGCAGCGGAGGTCACTAGACG |
| | | TGGGAGTGCATACTATATGTACTTGGACAGAAACGATGCTGGGGAGGCC |
| | | ATATCTTTTCCAACCACATTG |
| | | GGGATGAATAAGTGTTATATACAGATCATGGATCTTGGACACATGTGTG |
| | | ATGCCACCATGAGCTATGAAT |
| | | GCCCTATGCTGGATGAGGGGGTGGAACCAGATGACGTCGATTGTTGGTG |
| | | CAACACGACGTCAACTTGGGT |
| | | TGTGTACGGAACCTGCCATCACAAAAAAGGTGAAGCACGGAGATCTAG |
| | | AAGAGCTGTGACGCTCCCCTCC |
| | | CATTCCACTAGGAAGCTGCAAACGCGGTCGCAAACCTGGTTGGAATCAA |
| | | GAGAATACACAAAGCACTTGA |
| | | TTAGAGTCGAAAATTGGATATTCAGGAACCCTGGCTTCGCGTTAGCAGC |
| | | AGCTGCCATCGCTTGGCTTTT |
| | | GGGAAGCTCAACGAGCCAAAAAGTCATATACTTGGTCATGATACTGCTG |
| | | ATTGCCCCGGCATACAGCATC |
| | | AGGTGCATAGGAGTCAGCAATAGGGACTTTGTGGAAGGTATGTCAGGTG |
| | | GGACTTGGGTTGATATTGTCT |
| | | TGGAACATGGAGGTTGTGTCACCGTAATGGCACAGGACAAACCGACTGT |
| | | CGACATAGAGCTGGTTACAAC |
| | | AACAGTCAGCAACATGGCGGAGGTAAGATCCTACTGCTATGAGGCATCA |
| | | ATATCAGACATGGCTTCGGAC |
| | | AGCCGCTGCCCAACACAAGGTGAAGCCTACCTTGACAAGCAATCAGAC |
| | | ACTCAATATGTCTGCAAAAGAA |
| | | CGTTAGTGGACAGAGGCTGGGGAAATGGATGTGGACTTTTTGGCAAAGG |
| | | GAGTCTGGTGACATGCGCTAA |
| | | GTTTGCATGCTCCAAGAAAATGACCGGGAAGAGCATCCAGCCAGAGAA |
| | | TCTGGAGTACCGGATAATGCTG |
| | | TCAGTTCATGGCTCCCAGCACAGTGGGATGATCGTTAATGACACAGGAC |
| | | ATGAAACTGATGAGAATAGAG |
| | | CGAAGGTTGAGATAACGCCCAATTCACCAAGAGCCGAAGCCACCCTGG |
| | | GGGGTTTTGGAAGCCTAGGACT |
| | | TGATTGTGAACCGAGGACAGGCCTTGACTTTTCAGATTTGTATTACTTGA |
| | | CTATGAATAACAAGCACTGG |
| | | TTGGTTCACAAGGAGTGGTTCCACGACATTCCATTACCTTGGCACGCTG |
| | | GGGCAGACACCGGAACTCCAC |
| | | ACTGGAACAACAAAGAAGCACTGGTAGAGTTCAAGGACGCACATGCCA |
| | | AAAGGCAAACTGTCGTGGTTCT |
| | | AGGGAGTCAAGAAGGAGCAGTTCACACGGCCCTTGCTGGAGCTCTGGA |
| | | GGCTGAGATGGATGGTGCAAAG |
| | | GGAAGGCTGTCCTCTGGCCACTTGAAATGTCGCCTGAAAATGGATAAAC |
| | | TTAGATTGAAGGGCGTGTCAT |
| | | ACTCCTTGTGTACCGCAGCGTTCACATTCACCAAGATCCCGGCTGAAAC |
| | | ACTGCACGGGACAGTCACAGT |
| | | GGAGGTACAGTACGCAGGGACAGATGGACCTTGCAAGGTTCCAGCTCA |
| | | GATGGCGGTGGACATGCAAACT |
| | | CTGACCCCAGTTGGGAGGTTGATAACCGCTAACCCCGTAATCACTGAAA |
| | | GCACTGAGAACTCTAAGATGA |
| | | TGCTGGAACTTGATCCACCATTTGGGGACTCTTACATTGTCATAGGAGTC |
| | | GGGGAGAAGAAGATCACCCA |
| | | CCACTGGCACAGGAGTGGCAGCACCATTGGAAAAGCATTTGAAGCCACT |
| | | GTGAGAGGTGCCAAGAGAATG |
| | | GCAGTCTTGGGAGACACAGCCTGGGACTTTGGATCAGTTGGAGGCGCTC |
| | | TCAACTCATTGGGCAAGGGCA |
| | | TCCATCAAATTTTTGGAGCAGCTTTCAAATCATTGTTTGGAGGAATGTCC |
| | | TGGTTCTCACAAATTCTCAT |
| | | TGGAACGTTGCTGATGTGGTTGGGTCTGAACACAAAGAATGGATCTATT |
| | | TCCCTTATGTGCTTGGCCTTA |
| | | GGGGGAGTGTTGATCTTCTTATCCACAGCCGTCTCTGCTGATGTGGGGTG |
| | | CTCGGTGGACTTCTCAAAGA |
| | | AGGAGACGAGATGCGGTACAGGGGTGTTCGTCTATAACGACGTTGAAG |
| | | CCTGGAGGGACAGGTACAAGTA |
| | | CCATCCTGACTCCCCCCGTAGATTGGCAGCAGCAGTCAAGCAAGCCTGG |
| | | GAAGATGGTATCTGCGGGATC |
| | | TCCTCTGTTTCAAGAATGGAAAACATCATGTGGAGATCAGTAGAAGGGG |
| | | AGCTCAACGCAATCCTGGAAG |
| | | AGAATGGAGTTCAACTGACGGTCGTTGTGGGATCTGTAAAAAACCCCAT |
| | | GTGGGGAGGTCCACAGAGATT |
| | | GCCCGTGCCTGTGAACGAGCTGCCCCACGGCTGGAAGGCTTGGGGGAA |
| | | ATCGCACTTCGTCAGAGCAGCA |
| | | AAGACAAATAACAGCTTTGTCGTGGATGGTGACACACTGAAGGAATGCC |
| | | CACTCAAACATAGAGCATGGA |
| | | ACAGCTTTCTTGTGGAGGATCATGGGTTCGGGGTATTTCACACTAGTGTC |
| | | TGGCTCAAGGTTAGAGAAGA |
| | | TTATTCATTAGAGTGTGATCCAGCCGTTATTGGAACAGCTGTTAAGGGA |
| | | AAGGAGGCTGTACACAGTGAT |
| | | CTAGGCTACTGGATTGAGAGTGAGAAGAATGACACATGGAGGCTGAAG |
| | | AGGGCCCATCTGATCGAGATGA |
| | | AAACATGTGAATGGCCAAAGTCCCACACATTGTGGACAGATGGAATAG |
| | | AAGAGAGTGATCTGATCATACC |
| | | CAAGTCTTTAGCTGGGCCACTCAGCCATCACAATACCAGAGAGGGCTAC |
| | | AGGACCCAAATGAAAGGGCCA |
| | | TGGCACAGTGAAGAGCTTGAAATTCGGTTTGAGGAATGCCCAGGCACTA |
| | | AGGTCCACGTGGAGGAAACAT |
| | | GTGGAACAAGAGGACCATCTCTGAGATCAACCACTGCAAGCGGAAGGG |
| | | TGATCGAGGAATGGTGCTGCAG |
| | | GGAGTGCACAATGCCCCCACTGTCGTTCCGGGCTAAAGATGGCTGTTGG |
| | | TATGGAATGGAGATAAGGCCC |
| | | AGGAAAGAACCAGAAAGCAACTTAGTAAGGTCAATGGTGACTGCAGGA |
| | | TCAACTGATCACATGGATCACT |
| | | TCTCCCTTGGAGTGCTTGTGATTCTGCTCATGGTGCAGGAAGGGCTGAA |
| | | GAAGAGAATGACCACAAAGAT |
| | | CATCATAAGCACATCAATGGCAGTGCTGGTAGCTATGATCCTGGGAGGA |
| | | TTTTCAATGAGTGACCTGGCT |
| | | AAGCTTGCAATTTTGATGGGTGCCACCTTCGCGGAAATGAACACTGGAG |
| | | GAGATGTAGCTCATCTGGCGC |
| | | TGATAGCGGCATTCAAAGTCAGACCAGCGTTGCTGGTATCTTTCATCTTC |
| | | AGAGCTAATTGGACACCCCG |
| | | TGAAAGCATGCTGCTGGCCTTGGCCTCGTGTCTTTTGCAAACTGCGATCT |
| | | CCGCCTTGGAAGGCGACCTG |
| | | ATGGTTCTCATCAATGGTTTTGCTTTGGCCTGGTTGGCAATACGAGCGAT |
| | | GGTTGTTCCACGCACTGATA |
| | | ACATCACCTTGGCAATCCTGGCTGCTCTGACACCACTGGCCCGGGGCAC |
| | | ACTGCTTGTGGCGTGGAGAGC |
| | | AGGCCTTGCTACTTGCGGGGGGTTTATGCTCCTCTCTCTGAAGGGAAAA |
| | | GGCAGTGTGAAGAAGAACTTA |
| | | CCATTTGTCATGGCCCTGGGACTAACCGCTGTGAGGCTGGTCGACCCCA |
| | | TCAACGTGGTGGGGCTGCTGT |
| | | TGCTCACAAGGAGTGGGAAGCGGAGCTGGCCCCCTAGCGAAGTACTCA |
| | | CAGCTGTTGGCCTGATATGCGC |
| | | ATTGGCTGGAGGGTTCGCCAAGGCAGATATAGAGATGGCTGGGCCCATG |
| | | GCCGCGGTCGGTCTGCTAATT |
| | | GTCAGTTACGTGGTCTCAGGAAAGAGTGTGGACATGTACATTGAAAGAG |
| | | CAGGTGACATCACATGGGAAA |
| | | AAGATGCGGAAGTCACTGGAAACAGTCCCCGGCTCGATGTGGCGCTAG |
| | | ATGAGAGTGGTGATTTCTCCCT |
| | | GGTGGAGGATGACGGTCCCCCCATGAGAGAGATCATACTCAAGGTGGTC |
| | | CTGATGACCATCTGTGGCATG |
| | | AACCCAATAGCCATACCCTTTGCAGCTGGAGCGTGGTACGTATACGTGA |
| | | AGACTGGAAAAAGGAGTGGTG |
| | | CTCTATGGGATGTGCCTGCTCCCAAGGAAGTAAAAAAGGGGGAGACCA |
| | | CAGATGGAGTGTACAGAGTAAT |
| | | GACTCGTAGACTGCTAGGTTCAACACAAGTTGGAGTGGGAGTTATGCAA |
| | | GAGGGGGTCTTTCACACTATG |
| | | TGGCACGTCACAAAAGGATCCGCGCTGAGAAGCGGTGAAGGGAGACTT |
| | | GATCCATACTGGGGAGATGTCA |
| | | AGCAGGATCTGGTGTCATACTGTGGTCCATGGAAGCTAGATGCCGCCTG |
| | | GGACGGGCACAGCGAGGTGCA |
| | | GCTCTTGGCCGTGCCCCCCGGAGAGAGAGCGAGGAACATCCAGACTCTG |
| | | CCCGGAATATTTAAGACAAAG |
| | | GATGGGGACATTGGAGCGGTTGCGCTGGATTACCCAGCAGGAACTTCAG |
| | | GATCTCCAATCCTAGACAAGT |
| | | GTGGGAGAGTGATAGGACTTTATGGCAATGGGGTCGTGATCAAAAATG |
| | | GGAGTTATGTTAGTGCCATCAC |
| | | CCAAGGGAGGAGGGAGGAAGAGACTCCTGTTGAGTGCTTCGAGCCTTC |
| | | GATGCTGAAGAAGAAGCAGCTA |
| | | ACTGTCTTAGACTTGCATCCTGGAGCTGGGAAAACCAGGAGAGTTCTTC |
| | | CCGAAATAGTCCGTGAAGCCA |
| | | TAAAAACAAGACTCCGTACTGTGATCTTAGCTCCAACCAGGGTTGTCGC |
| | | TGCTGAAATGGAGGAAGCCCT |
| | | TAGAGGGCTTCCAGTGCGTTATATGACAACAGCAGTCAATGTCACCCAC |
| | | TCTGGAACAGAAATCGTCGAC |
| | | TTAATGTGCCATGCCACCTTCACTTCACGTCTACTACAGCCAATCAGAGT |
| | | CCCCAACTATAATCTGTATA |
| | | TTATGGATGAGGCCCACTTCACAGATCCCTCAAGTATAGCAGCAAGAGG |
| | | ATACATTTCAACAAGGGTTGA |
| | | GATGGGCGAGGCGGCTGCCATCTTCATGACCGCCACGCCACCAGGAACC |
| | | CGTGACGCATTTCCGGACTCC |
| | | AACTCACCAATTATGGACACCGAAGTGGAAGTCCCAGAGAGAGCCTGG |
| | | AGCTCAGGCTTTGATTGGGTGA |
| | | CGGATTATTCTGGAAAAACAGTTTGGTTTGTTCCAAGCGTGAGGAACGG |
| | | CAATGAGATCGCAGCTTGTCT |
| | | GACAAAGGCTGGAAAACGGGTCATACAGCTCAGCAGAAAGACTTTTGA |
| | | GACAGAGTTCCAGAAAACAAAA |
| | | CATCAAGAGTGGGACTTTGTCGTGACAACTGACATTTCAGAGATGGGCG |
| | | CCAACTTTAAAGCTGACCGTG |
| | | TCATAGATTCCAGGAGATGCCTAAAGCCGGTCATACTTGATGGCGAGAG |
| | | AGTCATTCTGGCTGGACCCAT |
| | | GCCTGTCACACATGCCAGCGCTGCCCAGAGGAGGGGGCGCATAGGCAG |
| | | GAATCCCAACAAACCTGGAGAT |
| | | GAGTATCTGTATGGAGGTGGGTGCGCAGAGACTGACGAAGACCATGCA |
| | | CACTGGCTTGAAGCAAGAATGC |
| | | TCCTTGACAATATTTACCTCCAAGATGGCCTCATAGCCTCGCTCTATCGA |
| | | CCTGAGGCCGACAAAGTAGC |
| | | AGCCATTGAGGGAGAGTTCAAGCTTAGGACGGAGCAAAGGAAGACCTT |
| | | TGTGGAACTCATGAAAAGAGGA |
| | | GATCTTCCTGTTTGGCTGGCCTATCAGGTTGCATCTGCCGGAATAACCTA |
| | | CACAGATAGAAGATGGTGCT |
| | | TTGATGGCACGACCAACAACACCATAATGGAAGACAGTGTGCCGGCAG |
| | | AGGTGTGGACCAGACACGGAGA |
| | | GAAAAGAGTGCTCAAACCGAGGTGGATGGACGCCAGAGTTTGTTCAGA |
| | | TCATGCGGCCCTGAAGTCATTC |
| | | AAGGAGTTTGCCGCTGGGAAAAGAGGAGCGGCTTTTGGAGTGATGGAA |
| | | GCCCTGGGAACACTGCCAGGAC |
| | | ACATGACAGAGAGATTCCAGGAAGCCATTGACAACCTCGCTGTGCTCAT |
| | | GCGGGCAGAGACTGGAAGCAG |
| | | GCCTTACAAAGCCGCGGCGGCCCAATTGCCGGAGACCCTAGAGACCATT |
| | | ATGCTTTTGGGGTTGCTGGGA |
| | | ACAGTCTCGCTGGGAATCTTTTTCGTCTTGATGAGGAACAAGGGCATAG |
| | | GGAAGATGGGCTTTGGAATGG |
| | | TGACTCTTGGGGCCAGCGCATGGCTCATGTGGCTCTCGGAAATTGAGCC |
| | | AGCCAGAATTGCATGTGTCCT |
| | | CATTGTTGTGTTCCTATTGCTGGTGGTGCTCATACCTGAGCCAGAAAAGC |
| | | AAAGATCTCCCCAGGACAAC |
| | | CAAATGGCAATCATCATCATGGTAGCAGTAGGTCTTCTGGGCTTGATTA |
| | | CCGCCAATGAACTCGGATGGT |
| | | TGGAGAGAACAAAGAGTGACCTAAGCCATCTAATGGGAAGGAGAGAGG |
| | | AGGGGGCAACCATGGGATTCTC |
| | | AATGGACATTGACCTGCGGCCAGCCTCAGCTTGGGCCATCTATGCTGCC |
| | | TTGACAACTTTCATTACCCCA |
| | | GCCGTCCAACATGCAGTGACCACTTCATACAACAACTACTCCTTAATGG |
| | | CGATGGCCACGCAAGCTGGAG |
| | | TGTTGTTTGGTATGGGCAAAGGGATGCCATTCTACGCATGGGACTTTGG |
| | | AGTCCCGCTGCTAATGATAGG |
| | | TTGCTACTCACAATTAACGCCCCTGACCCTAATAGTGGCCATCATTTTGC |
| | | TCGTGGCGCACTACATGTAC |
| | | TTGATCCCAGGGCTGCAGGCAGCAGCTGCGCGTGCTGCCCAGAAGAGA |
| | | ACGGCAGCTGGCATCATGAAGA |
| | | ACCCTGTTGTGGATGGAATAGTGGTGACTGACATTGACACAATGACAAT |
| | | TGACCCCCAAGTGGAGAAAAA |
| | | GATGGGACAGGTGCTACTCATGGCAGTAGCCGTCTCCAGCGCCATACTG |
| | | TCGCGGACCGCCTGGGGGTGG |
| | | GGGGAGGCTGGGGCCCTGATCACAGCCGCAACTTCCACTTTGTGGGAAG |
| | | GCTCTCCGAACAAGTACTGGA |
| | | ACTCCTCTACAGCCACTTCACTGTGTAACATTTTTAGGGGAAGTTACTTG |
| | | GCTGGAGCTTCTCTAATCTA |
| | | CACAGTGACAAGAAACGCTGGCTTGGTCAAGAGACGTGGGGGTGGAAC |
| | | AGGAGAGACCCTGGGAGAGAAA |
| | | TGGAAGGCCCGCTTGAACCAGATGTCGGCCCTGGAGTTCTACTCCTACA |
| | | AAAAGTCAGGCATCACCGAGG |
| | | TGTGCAGAGAAGAGGCCCGCCGCGCCCTCAAGGACGGTGTGGCAACGG |
| | | GAGGCCATGCTGTGTCCCGAGG |
| | | AAGTGCAAAGCTGAGATGGTTGGTGGAGCGGGGATACCTGCAGCCCTAT |
| | | GGAAAGGTCATTGATCTTGGA |
| | | TGTGGCAGAGGGGGCTGGAGTTACTACGCCGCCACCATCCGCAAAGTTC |
| | | AAGAAGTGAAAGGATACACAA |
| | | AAGGAGGCCCTGGTCATGAAGAACCCGTGTTGGTGCAAAGCTATGGGTG |
| | | GAACATAGTCCGTCTTAAGAG |
| | | TGGGGTGGACGTCTTTCATATGGCGGCTGAGCCGTGTGACACGTTGCTG |
| | | TGTGACATAGGTGAGTCATCA |
| | | TCTAGTCCTGAAGTGGAAGAAGCACGGACGCTCAGAGTCCTCTCCATGG |
| | | TGGGGGATTGGCTTGAAAAAA |
| | | GACCAGGAGCCTTTTGTATAAAAGTGTTGTGCCCATACACCAGCACTAT |
| | | GATGGAAACCCTGGAGCGACT |
| | | GCAGCGTAGGTATGGGGGAGGACTGGTCAGAGTGCCACTCTCCCGCAAC |
| | | TCTACACATGAGATGTACTGG |
| | | GTCTCTGGAGCGAAAAGCAACACCATAAAAAGTGTGTCCACCACGAGC |
| | | CAGCTCCTCTTGGGGCGCATGG |
| | | ACGGGCCTAGGAGGCCAGTGAAATATGAGGAGGATGTGAATCTCGGCT |
| | | CTGGCACGCGGGCTGTGGTAAG |
| | | CTGCGCTGAAGCTCCCAACATGAAGATCATTGGTAACCGCATTGAAAGG |
| | | ATCCGCAGTGAGCACGCGGAA |
| | | ACGTGGTTCTTTGACGAGAACCACCCATATAGGACATGGGCTTACCATG |
| | | GAAGCTATGAGGCCCCCACAC |
| | | AAGGGTCAGCGTCCTCTCTAATAAACGGGGTTGTCAGGCTCCTGTCAAA |
| | | ACCCTGGGATGTGGTGACTGG |
| | | AGTCACAGGAATAGCCATGACCGACACCACACCGTATGGTCAGCAAAG |
| | | AGTTTTCAAGGAAAAAGTGGAC |
| | | ACTAGGGTGCCAGACCCCCAAGAAGGTACTCGTCAGGTTATGAGCATGG |
| | | TCTCTTCCTGGTTGTGGAAAG |
| | | AGCTAGGCAAACACAAACGGCCACGAGTCTGTACCAAAGAAGAGTTCA |
| | | TCAACAAGGTTCGTAGCAATGC |
| | | AGCATTAGGGGCAATATTTGAAGAGGAAAAAGAGTGGAAGACTGCAGT |
| | | GGAAGCTGTGAACGATCCAAGG |
| | | TTCTGGGCTCTAGTGGACAAGGAAAGAGAGCACCACCTGAGAGGAGAG |
| | | TGCCAGAGTTGTGTGTACAACA |
| | | TGATGGGAAAAAGAGAAAAGAAACAAGGGGAATTTGGAAAGGCCAAG |
| | | GGCAGCCGCGCCATCTGGTATAT |
| | | GTGGCTAGGGGCTAGATTTCTAGAGTTCGAAGCCCTTGGATTCTTGAAC |
| | | GAGGATCACTGGATGGGGAGA |
| | | GAGAACTCAGGAGGTGGTGTTGAAGGGCTGGGATTACAAAGACTCGGA |
| | | TATGTCCTAGAAGAGATGAGTC |
| | | GCATACCAGGAGGAAGGATGTATGCAGATGACACTGCTGGCTGGGACA |
| | | CCCGCATCAGCAGGTTTGATCT |
| | | GGAGAATGAAGCTCTAATCACCAACCAAATGGAGAAAGGGCACAGGGC |
| | | CTTGGCATTGGCCATAATCAAG |
| | | TACACATACCAAAACAAAGTGGTAAAGGTCCTTAGACCAGCTGAAAAA |
| | | GGGAAAACAGTTATGGACATTA |
| | | TTTCGAGACAAGACCAAAGGGGGAGCGGACAAGTTGTCACTTACGCTCT |
| | | TAACACATTTACCAACCTAGT |
| | | GGTGCAACTCATTCGGAATATGGAGGCTGAGGAAGTCCTAGAGATGCA |
| | | AGACTTGTGGCTGCTGCGGAGG |
| | | TCAGAGAAAGTGACCAACTGGTTGCAGAGCAACGGATGGGATAGGCTC |
| | | AAACGAATGGCAGTCAGTGGAG |
| | | ATGATTGCGTTGTGAAGCCAATTGATGATAGGTTTGCACATGCCCTCAG |
| | | GTTCTTGAATGATATGGGAAA |
| | | AGTTAGGAAGGACACACAAGAGTGGAAACCCTCAACTGGATGGGACAA |
| | | CTGGGAAGAAGTTCCGTTTTGC |
| | | TCCCACCACTTCAACAAGCTCCATCTCAAGGACGGGAGGTCCATTGTGG |
| | | TTCCCTGCCGCCACCAAGATG |
| | | AACTGATTGGCCGGGCCCGCGTCTCTCCAGGGGCGGGATGGAGCATCCG |
| | | GGAGACTGCTTGCCTAGCAAA |
| | | ATCATATGCGCAAATGTGGCAGCTCCTTTATTTCCACAGAAGGGACCTC |
| | | CGACTGATGGCCAATGCCATT |
| | | TGTTCATCTGTGCCAGTTGACTGGGTTCCAACTGGGAGAACTACCTGGTC |
| | | AATCCATGGAAAGGGAGAAT |
| | | GGATGACCACTGAAGACATGCTTGTGGTGTGGAACAGAGTGTGGATTGA |
| | | GGAGAACGACCACATGGAAGA |
| | | CAAGACCCCAGTTACGAAATGGACAGACATTCCCTATTTGGGAAAAAGG |
| | | GAAGACTTGTGGTGTGGATCT |
| | | CTCATAGGGCACAGACCGCGCACCACCTGGGCTGAGAACATTAAAAAC |
| | | ACAGTCAACATGGTGCGCAGGA |
| | | TCATAGGTGATGAAGAAAAGTACATGGACTACCTATCCACCCAAGTTCG |
| | | CTACTTGGGTGAAGAAGGGTC |
| | | TACACCTGGAGTGCTGTAAGCACCAATCTTAATGTTGTCAGGCCTGCTA |
| | | GTCAGCCACAGCTTGGGGAAA |
| | | GCTGTGCAGCCTGTGACCCCCCCAGGAGAAGCTGGGAAACCAAGCCTAT |
| | | AGTCAGGCCGAGAACGCCATG |
| | | GCACGGAAGAAGCCATGCTGCCTGTGAGCCCCTCAGAGGACACTGAGTC |
| | | AAAAAACCCCACGCGCTTGGA |
| | | GGCGCAGGATGGGAAAAGAAGGTGGCGACCTTCCCCACCCTTCAATCTG |
| | | GGGCCTGAACTGGAGATCAGC |
| | | TGTGGATCTCCAGAAGAGGGACTAGTGGTTAGAGGAGACCCCCCGGAA |
| | | AACGCAAAACAGCATATTGACG |
| | | CTGGGAAAGACCAGAGACTCCATGAGTTTCCACCACGCTGGCCGCCAGG |
| | | CACAGATCGCCGAATAGCGGC |
| | | GGCCGGTGTGGGGAAATCCATGGGTCT |
| 2 | Zika virus | MKNPKKKSGGFRIVNMLKRGVARVSPEGGLKRLPAGLLLGHGPIRMVLAIL |
| | isolate | AFLRFTAIKPSLGLINRWG |
| | ZIKV/H. | SVGKKEAMEIIKKFKKDLAAMLRIINARKEKKRRGADTSVGIVGLLLTTAM |
| | sapiens/ | AAEVTRRGSAYYMYLDRND |
| | Brazil/ | AGEAISEPTTLGMNKCYIQIMDLGHMCDATMSYECPMLDEGVEPDDVDCW |
| | PE243/2015, | CNTTSTWVVYGTCHTIKKGEA |
| | polyprotein | RRSRRAVTLPSHSTRKLQTRSQTWLESREYTKHLIRVENWIERNPGFALAAA |
| | | AIAWLLGSSTSQKVIYLV |
| | | MILLIAPAYSIRCIGVSNRDEVEGMSGGTWVDIVLEHGGCVTVMAQDKPTV |
| | | DIELVTTTVSNMAEVRSYC |
| | | YEAISDMASDSRCPTQGEAYLDKQSDTQYVCKRTLVDRGWGNGCGLFGK |
| | | GSLVTCAKFACSKKMTGKSI |
| | | QPENLEYRIMLSVHGSQHSGMIVNDTGHETDENRAKVEITPNSPRAEATLG |
| | | GFGSLGLDCEPRTGLDFSD |
| | | LYYLTMNNKHWLVHKEWEHDIPLPWHAGADTGTPFIWNNKEALVEFKDA |
| | | HAKRQTVVVLGSQEGAVHTALA |
| | | GALEAEMDGAKGRLS SGHLKCRLKMDKLRLKGVSYSLCTAAFTFTKIPAET |
| | | LHGTVTVEVQYAGTDGPCK |
| | | VPAQMAVDMQTLTPVGRLITANPVITESTENSKMMLELDPPFGDSYIVIGVG |
| | | EKKITHHWHRSGSTIGKA |
| | | FEATVRGAKRMAVLGDTAWDEGSVGGALNSLGKGIFIQIFGAAFKSLEGGM |
| | | SWFSQILIGTLLMWLGLNTK |
| | | NGSISLMCLALGGVLIFLSTAVSADVGCSVDFSKKETRCGTGVEVYNDVEA |
| | | WRDRYKYHPDSPRRLAAAV |
| | | KQAWEDGICGISSVSRMENIMWRSVEGELNAILEENGVQLTVVVGSVKNP |
| | | MWGGPQRLPVPVNELPHGWK |
| | | AWGKSHEVRAAKTNNSFVVDGDTLKECPLKHRAWNSFLVEDHGEGVEHTS |
| | | VWLKVREDYSLECDPAVIGT |
| | | AVKGKEAVHSDLGYWIESEKNDTWRLKRAHLIEMKTCEWPKSHTLWTDGI |
| | | EESDLIIPKSLAGPLSHHNT |
| | | REGYRTQMKGPWHSEELEIRFEECPGTKVHVEETCGTRGP SLRSTTASGRVI |
| | | EEWCCRECTMPPLSFRAK |
| | | DGCWYGMEIRPRKEPESNLVRSMVTAGSTDHMDHFSLGVLVILLMVQEGL |
| | | KKRMTTKIIISTSMAVLVAM |
| | | ILGGFSMSDLAKLAILMGATFAEMNTGGDVAHLALIAAFKVRPALLVSFIFR |
| | | ANWTPRESMLLALASCLL |
| | | QTAISALEGDLMVLINGFALAWLAIRAMANPRTDNITLAILAALTPLARGTL |
| | | LVAWRAGLATCGGFMLLS |
| | | LKGKGSVKKNLPFVMALGLTAVRLVDPINVVGLLLLTRSGKRSWPPSEVLT |
| | | AVGLICALAGGFAKADIEM |
| | | AGPMAAVGLLIVSYVVSGKSVDMYIERAGDMVEKDAEVTGNSPRLDVAL |
| | | DESGDFSLVEDDGPPMREII |
| | | LKVVLMTICGMNPIAIPFAAGAWYVYVKTGKRSGALWDVPAPKEVKKGET |
| | | TDGVYRVMTRRLLGSTQVGV |
| | | GVMQEGVEHTMWHVTKGSALRSGEGRLDPYWGDVKQDLVSYCGPWKLD |
| | | AAWDGHSEVQLLAVPPGERARN |
| | | IQTLPGIEKTKDGDIGAVALDYPAGTSGSPILDKCGRVIGLYGNGVVIKNGS |
| | | YVSAITQGRREEETPVEC |
| | | FEPSMLKKKQLTVLDLHPGAGKTRRVLPEIVREAIKTRLRTVILAPTRVVAA |
| | | EMEEALRGLPVRYMTTAV |
| | | NVTHSGTEIVDLMCHATFTSRLLQPIRVPNYNLYIMDEAHFTDPSSIAARGYI |
| | | STRVEMGEAAAIFMTAT |
| | | PPGTRDAFPDSNSPIMDTEVEVPERAWS SGFDWVTDYSGKTVWFVPSVRNG |
| | | NEIAACLTKAGKRVIQLSR |
| | | KTFETEFQKTKHQEWDFVVTTDISEMGANFKADRVIDSRRCLKPVILDGER |
| | | VILAGPMPVTHASAAQRRG |
| | | RIGRNPNKPGDEYLYGGGCAETDEDHAHWLEARMLLDNIYLQDGLIASLY |
| | | RPEADKVAAIEGEFKLRTEQ |
| | | RKTFVELMKRGDLPVWLAYQVASAGITYTDRRWCEDGTTNNTIMEDSVPA |
| | | EVWTRHGEKRVLKPRWMDAR |
| | | VCSDHAALKSEKEFAAGKRGAAFGVMEALGTLPGHMTERFQEAIDNLAVL |
| | | MRAETGSRPYKAAAAQLPET |
| | | LETIMLLGLLGTVSLGIFFVLMRNKGIGKMGEGMVTLGASAWLMWLSEIEP |
| | | ARIACVLIVVFLLLVVLIP |
| | | EPEKQRSPQDNQMAIIIMVAVGLLGLITANELGWLERTKSDLSHLMGRREE |
| | | GATMGFSMDIDLRPASAWA |
| | | IYAALTTFITPAVQHAVTTSYNNYSLMAMATQAGVLFGMGKGMPFYAWD |
| | | FGVPLLMIGCYSQLTPLTLIV |
| | | AIILLVAHYMYLIPGLQAAAARAAQKRTAAGIMKNPVVDGIVVTDIDTMTI |
| | | DPQVEKKMGQVLLMAVAVS |
| | | SAILSRTAWGWGEAGALITAATSTLWEGSPNKYWNSSTATSLCNIERGSYL |
| | | AGASLIYTVTRNAGLVKRR |
| | | GGGTGETLGEKWKARLNQMSALEFYSYKKSGITEVCREEARRALKDGVAT |
| | | GGHAVSRGSAKLRWLVERGY |
| | | LQPYGKVIDLGCGRGGWSYYAATIRKVQEVKGYTKGGPGHEEPVLVQSYG |
| | | WNIVRLKSGVDVFHMAAEPC |
| | | DTLLCDIGESSSSPEVEEARTLRVLSMVGDWLEKRPGAFCIKVLCPYTSTMM |
| | | ETLERLQRRYGGGLVRVP |
| | | LSRNSTHEMYWVSGAKSNTIKSVSTTSQLLLGRMDGPRRPVKYEEDVNLGS |
| | | GTRAVVSCAEAPNMKIIGN |
| | | RIERIRSEHAETWFFDENHPYRTWAYHGSYEAPTQGSASSLINGVVRLLSKP |
| | | WDVVTGVTGIAMTDTTPY |
| | | GQQRVFKEKVDTRVPDPQEGTRQVMSMVSSWLWKELGKHKRPRVCTKEE |
| | | FINKVRSNAALGAIFEEEKEW |
| | | KTAVEAVNDPRFWALVDKEREHHLRGECQSCVYNMMGKREKKQGEFGK |
| | | AKGSRAIWYMWLGARFLEFEAL |
| | | GELNEDHWMGRENSGGGVEGLGLQRLGYVLEEMSRIPGGRMYADDTAGW |
| | | DTRISRFDLENEALITNQMEK |
| | | GHRALALAIIKYTYQNKVVKVLRPAEKGKTVMDIISRQDQRGSGQVVTYAL |
| | | NTFTNLVVQLIRNMEAEEV |
| | | LEMQDLWLLRRSEKVTNWLQSNGWDRLKRMAVSGDDCVVKPIDDRFAHA |
| | | LRFLNDMGKVRKDTQEWKPST |
| | | GWDNWEEVPFCSHHFNKLHLKDGRSIVVPCRHQDELIGRARVSPGAGWSIR |
| | | ETACLAKSYAQMWQLLYFH |
| | | RRDLRLMANAICSSVPVDWVPTGRTTWSIHGKGEWMTTEDMLVVWNRV |
| | | WIEENDHMEDKTPVTKWTDIPY |
| | | LGKREDLWCGSLIGHRPRTTWAENIKNTVNMVRRIIGDEEKYMDYLSTQVR |
| | | YLGEEGSTPGVL |

The preceding merely illustrates the principles of this disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of this disclosure and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary embodiments shown and described herein. Rather, the scope and spirit of the present disclosure is embodied by the appended claims.

The following non-limiting embodiments provide illustrative examples of the invention, but do not limit the scope of the invention.

### Embodiment 1

A method of promoting an immune response to cancer comprising administering to a subject in need thereof a viral composition comprising a flavivirus or a portion of the flavivirus.

### Embodiment 2

The method of embodiment 1, provided that the flavivirus or portion of the flavivirus comprises a danger signal that promotes the immune response to the cancer.

### Embodiment 3

The method of embodiment 1 or embodiment 2, provided that the flavivirus has tropism for cancer cells.

### Embodiment 4

The method of any of embodiments 1-3, provided that the flavivirus has tropism for dividing cells.

### Embodiment 5

The method of any of embodiments 1-4, provided that the viral composition targets the immune response to the cancer in the subject.

### Embodiment 6

The method of any of embodiments 1-5, provided that the viral composition causes mitotic impairment, cancer cell death, or both mitotic impairment and cancer cell death.

### Embodiment 7

The method of any of embodiments 1-6, provided that the viral composition causes preferential mitotic impairment to a cancer cell as compared to a non-cancer cell, adult stem cell, and/or post-mitotic cell.

### Embodiment 8

The method of any of embodiments 1-7, provided that the cancer comprises a solid tumor.

### Embodiment 9

The method of any of embodiments 1-8, provided that the cancer comprises a brain cancer.

### Embodiment 10

The method of embodiment 9, provided that the brain cancer is astrocytoma, oligodendroglioma, ependymoma, meningioma, schwannoma, craniopharyngioma, germinoma, pineocytoma, or a combination thereof.

### Embodiment 11

The method of any of embodiments 1-10, provided that administering comprises intraperitoneal injection, intracranial injection, intratumoral injection, systemic administration, parenteral administration, intravenous injection, intrathecal administration, intravenous administration, or a combination thereof.

### Embodiment 12

The method of any of embodiments 1-11, provided that the flavivirus comprises Zika virus, spondweni virus, kedougous virus, or a combination thereof.

### Embodiment 13

The method of any of embodiments 1-12, provided that the portion of the flavivirus comprises at least about 10, 20, 30, 40, 50, 80, 100, 150, or 200 nucleic acids having at least about 90% sequence identity to a nucleic acid sequence of the flavivirus.

### Embodiment 14

The method of any of embodiments 1-13, provided that the viral composition comprises a nucleic acid sequence at least about 20, 50, 100, 150, 200, 300, or 500 nucleic acids in length, having at least about 70%, 75%, 80%, 85%, 90%, or 95% identity to a nucleic acid sequence of a Zika virus.

### Embodiment 15

The method of any of embodiments 1-14, provided that the viral composition comprises a nucleic acid sequence at least about 20, 50, 100, 150, 200, 300, or 500 nucleic acids in length, having at least about 70%, 75%, 80%, 85%, 90%, or 95% identity to a sequence of SEQ ID NO: 1.

### Embodiment 16

The method of any of embodiments 1-15, provided that the flavivirus or portion of the flavivirus comprises a nucleic acid sequence encoding for a capsid protein of the flavivirus, a membrane protein of the flavivirus, an envelope protein of the flavivirus, a nonstructural (NS) protein of the flavivirus, or a combination thereof.

### Embodiment 17

The method of embodiment 16, provided that the capsid protein comprises an amino acid sequence at least about 70%, 75%, 80%, 85%, 90%, or 95% identical to the amino acid sequence 1-122 of SEQ ID NO: 2.

### Embodiment 18

The method of embodiment 16 or embodiment 17, provided that the membrane protein comprises an amino acid sequence at least about 70%, 75%, 80%, 85%, 90%, or 95% identical to the amino acid sequence 216-290 of SEQ ID NO: 2.

### Embodiment 19

The method of any of embodiments 16-18, provided that the envelope protein comprises an amino acid sequence at least about 70%, 75%, 80%, 85%, 90%, or 95% identical to the amino acid sequence 795-1146 of SEQ ID NO: 2.

### Embodiment 20

The method of any of embodiments 16-19, provided that the NS protein comprises an amino acid sequence at least about 70%, 75%, 80%, 85%, 90%, or 95% identical to the amino acid sequence 1147-1372 of SEQ ID NO: 2.

### Embodiment 21

The method of any of embodiments 16-20, provided that the NS protein comprises an amino acid sequence at least about 70%, 75%, 80%, 85%, 90%, or 95% identical to the amino acid sequence 1373-1502 of SEQ ID NO: 2.

### Embodiment 22

The method of any of embodiments 16-21, provided that the NS protein comprises an amino acid sequence at least about 70%, 75%, 80%, 85%, 90%, or 95% identical to the amino acid sequence 1503-2119 of SEQ ID NO: 2.

### Embodiment 23

The method of any of embodiments 16-22, provided that the NS protein comprises an amino acid sequence at least about 70%, 75%, 80%, 85%, 90%, or 95% identical to the amino acid sequence 2120-2246 of SEQ ID NO: 2.

### Embodiment 24

The method of any of embodiments 16-23, provided that the NS protein comprises an amino acid sequence at least about 70%, 75%, 80%, 85%, 90%, or 95% identical to the amino acid sequence 2247-2269 of SEQ ID NO: 2.

### Embodiment 25

The method of any of embodiments 16-24, provided that the NS protein comprises an amino acid sequence at least about 70%, 75%, 80%, 85%, 90%, or 95% identical to the amino acid sequence 2521-3423 of SEQ ID NO: 2.

### Embodiment 26

The method of any of embodiments 16-25, provided that the NS protein comprises NS1, NS2A, NS2B, NS3, NS4A, NS4B, NS5, or a portion or combination thereof.

### Embodiment 27

The method of any of embodiments 1-26, further comprising administering to the subject an antiviral agent configured to kill the flavivirus.

### Embodiment 28

The method of embodiment 27, provided that the antiviral agent comprises a nucleoside analog.

### Embodiment 29

The method of embodiment 28, provided that the nucleoside analog comprises sofosbuvir, 2′-C-methyladenosine, or a combination thereof.

### Embodiment 30

The method of any of embodiments 1-29, provided that the subject is administered an immunostimulatory agent.

### Embodiment 31

The method of embodiment 30, provided that the viral composition is administered in combination with the immunostimulatory agent.

### Embodiment 32

The method of embodiment 30 or embodiment 31, provided that the viral composition comprises the immunostimulatory agent.

### Embodiment 33

The method of any of embodiments 30-32, provided that the flavivirus or portion of the flavivirus targets the immunostimulatory agent to the cancer.

### Embodiment 34

The method of any of embodiments 30-33, provided that the viral composition and the immunostimulatory agent provide a synergistic effect on the immune response to the cancer.

### Embodiment 35

The method of any of embodiments 30-34, provided that the viral composition and immunostimulatory agent are administered in a single composition.

### Embodiment 36

The method of any of embodiments 30, 31 or 33-35, provided that the viral composition and immunostimulatory agent are administered as separate compositions.

### Embodiment 37

The method of any of embodiments 30-36, provided that the immunostimulatory agent comprises a Toll-like receptor (TLR) agonist.

### Embodiment 38

The method of embodiment 37, provided that the Toll-like receptor agonist comprises lipopolysaccharide (LPS), imiquimod (TLR7 agonist), rintatolimod, resiquimod, or a combination thereof.

### Embodiment 39

The method of any of embodiments 30-38, provided that the immunostimulatory agent comprises granulocyte-macrophage colony-stimulating factor (GMCSF), heat shock protein (Hsp) 70, Hsp90, Hsp110, CpG oligonucleotide, or a combination thereof.

### Embodiment 40

The method of any of embodiments 30-39, provided that the immunostimulatory agent comprises an adjuvant.

### Embodiment 41

The method of embodiment 40, provided that the adjuvant comprises an aluminum salt.

### Embodiment 42

The method of embodiment 41, provided that the aluminum salt comprises aluminum phosphate, aluminum hydroxide, aluminum potassium sulfate, or a combination thereof.

### Embodiment 43

The method of any of embodiments 40-42, provided that the adjuvant comprises an oil-in-water emulsion.

### Embodiment 44

The method of embodiment 43, provided that the oil-in-water emulsion comprises squalene, polysorbate 80 (Tween 80), sorbitan trioleate, alpha-tocopherol, or a combination thereof.

### Embodiment 45

The method of embodiment 43 or embodiment 44, provided that the oil-in-water emulsion comprises MF59.

Embodiment 46 The method of any of embodiments 43-45, provided that the oil-in-water emulsion comprises AS03 (Adjuvant System 03).

### Embodiment 47

The method of any of embodiments 40-46, provided that the adjuvant comprises a liposome.

### Embodiment 48

The method of any of embodiments 40-47, provided that the adjuvant comprises a Toll-like receptor agonist.

### Embodiment 49

The method of any of embodiments 40-48, provided that the adjuvant comprises GLA-SE (glucopyranosyl lipid adjuvant formulated in a stable emulsion).

### Embodiment 50

The method of any of embodiments 40-49, provided that the adjuvant comprises a synthetic oligodeoxynucleotide (ODN1a).

### Embodiment 51

The method of any of embodiments 40-50, provided that the adjuvant comprises a peptide comprising KLKL(5)KLK (SEQ ID NO: 3).

### Embodiment 52

The method of any of embodiments 40-51, provided that the adjuvant comprises IC31®.

### Embodiment 53

The method of any of embodiments 40-52, provided that the adjuvant comprises dimethyldioctadecyl-ammonium (DDA).

### Embodiment 54

The method of any of embodiments 40-53, provided that the adjuvant comprises trehalose 6,6-dibehenate (TDB).

### Embodiment 55

The method of any of embodiments 40-54, provided that the adjuvant comprises CAF01.

### Embodiment 56

The method of any of embodiments 40-55, provided that the adjuvant comprises monophosphoryl lipid A.

### Embodiment 57

The method of any of embodiments 1-56, provided that the subject is treated with an additional cancer therapy.

### Embodiment 58

The method of embodiment 57, provided that the viral composition is administered in combination with the additional cancer therapy.

### Embodiment 59

The method of embodiment 57 or embodiment 58, provided that the viral composition comprises the additional cancer therapy.

### Embodiment 60

The method of any of embodiments 57-59, provided that the flavivirus or portion of the flavivirus targets the additional cancer therapy to the cancer.

### Embodiment 61

The method of any of embodiments 57-60, provided that the viral composition and the additional cancer therapy provide a synergistic effect on the immune response to the cancer.

### Embodiment 62

The method of any of embodiments 57-61, provided that the viral composition and additional cancer therapy are administered in a single composition.

### Embodiment 63

The method of any of embodiments 57, 58 or 60-62, provided that the viral composition and additional cancer therapy are administered as separate compositions.

### Embodiment 64

The method of any of embodiments 57-63, provided that the cancer therapy comprises an immune checkpoint inhibitor.

### Embodiment 65

The method of embodiment 64, provided that the immune checkpoint inhibitor comprises an anti-CTLA-4 antibody, an anti-PD-1 antibody, an anti-PD-1L antibody, or a combination thereof.

### Embodiment 66

The method of embodiment 65, provided that the anti-CTLA-4 antibody is ipilimumab or an antibody that binds to the same epitope as ipilimumab.

### Embodiment 67

The method of embodiment 65, provided that the anti-PD-1 antibody is pembrolizumab or an antibody that binds to the same epitope as pembrolizumab.

### Embodiment 68

The method of embodiment 65, provided that the anti-PD-1 antibody is nivolumab or an antibody that binds to the same epitope as nivolumab.

### Embodiment 69

The method of embodiment 65, provided that the anti-PD-1 antibody is BGB-A317 or an antibody that binds to the same epitope as BGB-A317.

### Embodiment 70

The method of embodiment 65, provided that the anti-PD-1L antibody is avelumab or an antibody that binds to the same epitope as avelumab.

### Embodiment 71

The method of embodiment 65, provided that the anti-PD-1L antibody is atezolizumab or an antibody that binds to the same epitope as atezolizumab.

### Embodiment 72

The method of embodiment 65, provided that the anti-PD-1L antibody is durvalumab or an antibody that binds to the same epitope as durvalumab.

### Embodiment 73

The method of any of embodiments 57-63, provided that the cancer therapy comprises adoptive T-cell therapy.

### Embodiment 74

The method of any of embodiments 57-63, provided that the cancer therapy comprises an anti-CD47 antibody.

### Embodiment 75

The method of any of embodiments 57-63, provided that the cancer therapy comprises administration of a cytokine.

### Embodiment 76

The method of embodiment 75, provided that the cytokine is an interferon.

### Embodiment 77

The method of embodiment 75, provided that the cytokine is an interleukin.

### Embodiment 78

The method of any of embodiments 57-63, provided that the cancer therapy comprises adoptive cell transfer.

### Embodiment 79

The method of embodiment 78, provided that the cancer therapy comprises chimeric antigen receptor (CAR) T-cell therapy.

### Embodiment 80

The method of any of embodiments 57-63, provided that the cancer therapy comprises radiation therapy.

### Embodiment 81

The method of any of embodiments 57-63, provided that the cancer therapy comprises chemotherapy.

### Embodiment 82

The method of embodiment 81, provided that the chemotherapy induces immunogenic cancer cell death.

### Embodiment 83

The method of embodiment 81 or embodiment 82, provided that the chemotherapy comprises doxorubicin and/or docetaxel.

### Embodiment 84

The method of any of embodiments 57-63, provided that the cancer therapy comprises surgery.

### Embodiment 85

The method of any of embodiments 1-84, provided that the flavivirus or portion of the flavivirus is genetically engineered.

### Embodiment 86

The method of embodiment 85, provided that the genetically engineered virus binds to a tumor-specific antigen of a cell from the cancer.

### Embodiment 87

The method of embodiment 86, provided that the tumor-specific antigen is EGFR or a portion or derivative thereof.

### Embodiment 88

The method of embodiment 87, provided that the portion or derivative of EGFR comprises EGFRvIII.

### Embodiment 89

The method of embodiment 85, provided that the genetically engineered virus binds to a tumor-associated antigen of a cell from the cancer.

### Embodiment 90

The method of any of embodiments 86-89, provided that the cell from the cancer is a brain cancer cell.

### Embodiment 91

The method of any of embodiments 85-90, provided that the engineered virus expresses IL-12.

### Embodiment 92

The method of any of embodiments 85-91, provided that the engineered virus expresses GMCSF.

### Embodiment 93

The method of any of embodiments 85-92, provided that the flavivirus is engineered to comprise a suicide gene expressible under an inducible promoter.

### Embodiment 94

The method of embodiment 93, further comprising administering to the subject the promoter or an activator thereof to express the suicide gene, thereby killing the flavivirus.

### Embodiment 95

The method of any of embodiments 1-94, provided that the viral composition comprises a recombinant binding domain having specificity for a cancer cell marker.

### Embodiment 96

The method of any of embodiments 1-95, provided that the flavivirus is engineered for selective proliferation in the cancer cell.

### Embodiment 97

The method of any of embodiments 1-96, provided that the flavivirus comprises a genetic element of a Zika virus genome that is not found in a dengue virus genome.

### Embodiment 98

A combination comprising a flavivirus or a portion of the flavivirus, and an immunostimulatory agent.

### Embodiment 99

The combination of embodiment 98, provided that the flavivirus or portion of the flavivirus and the immunostimulatory agent are provided in a single composition.

### Embodiment 100

The combination of embodiment 98, provided that the flavivirus or portion of the flavivirus and the immunostimulatory agent are provided in separate compositions.

### Embodiment 101

The combination of any of embodiments 98-100, provided that the immunostimulatory agent comprises a Toll-like receptor (TLR) agonist.

### Embodiment 102

The combination of embodiment 101, provided that the Toll-like receptor agonist comprises lipopolysaccharide (LPS), imiquimod (TLR7 agonist), rintatolimod, resiquimod, or a combination thereof.

### Embodiment 103

The combination of any of embodiments 98-102, provided that the immunostimulatory agent comprises granulocyte-macrophage colony-stimulating factor (GMCSF), heat shock protein (Hsp) 70, Hsp90, Hsp110, CpG oligonucleotide, or a combination thereof.

### Embodiment 104

The combination of any of embodiments 98-103, provided that the immunostimulatory agent comprises an adjuvant.

### Embodiment 105

The combination of embodiment 104, provided that the adjuvant comprises an aluminum salt.

### Embodiment 106

The combination of embodiment 105, provided that the aluminum salt comprises aluminum phosphate, aluminum hydroxide, aluminum potassium sulfate, or a combination thereof.

### Embodiment 107

The combination of any of embodiments 104-106, provided that the adjuvant comprises an oil-in-water emulsion.

### Embodiment 108

The combination of embodiment 107, provided that the oil-in-water emulsion comprises squalene, polysorbate 80 (Tween 80), sorbitan trioleate, alpha-tocopherol, or a combination thereof.

### Embodiment 109

The combination of embodiment 107 or embodiment 108, provided that the oil-in-water emulsion comprises MF59.

### Embodiment 110

The combination of any of embodiments 107-109, provided that the oil-in-water emulsion comprises AS03 (Adjuvant System 03).

### Embodiment 111

The combination of any of embodiments 104-110, provided that the adjuvant comprises a liposome.

### Embodiment 112

The combination of any of embodiments 104-111, provided that the adjuvant comprises a Toll-like receptor agonist.

### Embodiment 113

The combination of any of embodiments 104-112, provided that the adjuvant comprises GLA-SE (glucopyranosyl lipid adjuvant formulated in a stable emulsion).

### Embodiment 114

The combination of any of embodiments 104-113, provided that the adjuvant comprises a synthetic oligodeoxynucleotide (ODN1a).

### Embodiment 115

The combination of any of embodiments 104-114, provided that the adjuvant comprises a peptide comprising KLKL(5)KLK (SEQ ID NO: 3).

### Embodiment 116

The combination of any of embodiments 104-115, provided that the adjuvant comprises IC31®.

### Embodiment 117

The combination of any of embodiments 104-116, provided that the adjuvant comprises dimethyldioctadecyl-ammonium (DDA).

### Embodiment 118

The combination of any of embodiments 104-117, provided that the adjuvant comprises trehalose 6,6-dibehenate (TDB).

### Embodiment 119

The combination of any of embodiments 104-118, provided that the adjuvant comprises CAF01.

### Embodiment 120

The combination of any of embodiments 104-119, provided that the adjuvant comprises monophosphoryl lipid A.

### Embodiment 121

A method of treating cancer comprising administering the combination of any of embodiments 98-120 to a subject in need thereof.

### Embodiment 122

The method of embodiment 121, provided that the flavivirus or portion of the flavivirus targets the immunostimulatory agent to the cancer.

### Embodiment 123

The method of embodiment 121 or embodiment 122, provided that the flavivirus or the portion of the flavivirus, and the immunostimulatory agent provide a synergistic effect on an immune response to the cancer.

### Embodiment 124

The method of any of embodiments 121-123, provided that the flavivirus or portion of the flavivirus and the immunostimulatory agent are administered in a single composition.

### Embodiment 125

The method of any of embodiments 121-124, provided that the flavivirus or portion of the flavivirus and the immunostimulatory agent are administered as separate compositions.

### Embodiment 126

A method of treating a brain cancer comprising administering to a subject in need thereof a viral composition comprising a flavivirus or portion of a flavivirus wherein the flavivirus is engineered to comprise a heterologous nucleic acid sequence encoding a suicide gene.

### Embodiment 127

The method of embodiment 126, wherein the brain cancer is selected from the group consisting of astrocytoma, oligodendroglioma, ependymoma, meningioma, schwannoma, craniopharyngioma, germinoma, and pineocytoma.

### Embodiment 128

The method of any of embodiments 126-127, wherein the flavivirus comprises Zika virus, spondweni virus, kedougous virus, or a combination thereof.

### Embodiment 129

The method of any of embodiments 126-128, wherein the flavivirus comprises Zika virus.

### Embodiment 130

The method of any of embodiments 126-129, wherein the flavivirus comprises a nucleic acid sequence encoding for a capsid protein of the flavivirus, a membrane protein of the flavivirus, an envelope protein of the flavivirus, a nonstructural (NS) protein of the flavivirus, or a combination thereof.

### Embodiment 131

The method of any of embodiments 126-130, wherein the suicide gene is expressible under an inducible promoter.

### Embodiment 132

The method of embodiment 131, wherein the inducible promoter is cytomegalovirus (CMV).

### Embodiment 133

The method of any of embodiments 126-132, wherein the suicide gene encodes a protein that converts a prodrug into a cytotoxic agent.

### Embodiment 134

The method of embodiment 133, wherein the protein that converts a prodrug into the cytotoxic agent has enzymatic activity selected from the group consisting of thymidine kinase activity, cytosine deaminase activity, purine nucleoside phosphorylase activity, uracil phosphoribosyl transferase activity, and thymidylate kinase activity.

### Embodiment 135

The method of any of embodiments 133-134, wherein the prodrug is selected from the group consisting of ganciclovir, ganciclovir elaidic acid ester, penciclovir, acyclovir, valacyclovir, (E)-5-(2-bromovinyl)-2′deoxyuridine, zidovudine, 2′-exo-methanocarbathymidine, 5-fluorocytosine, 6-methylpurine deoxyriboside, fludarabine, 5-fluorocytosine, 5-fluorouracil, and azidothymidine.

### Embodiment 136

The method of any of embodiments 133-135, wherein the prodrug is administered to the subject.

### Embodiment 137

The method of any of embodiments 126-136, wherein the viral composition promotes an immune response to the brain cancer.

### Embodiment 138

The method of any of embodiments 126-137, wherein the viral composition causes preferential mitotic impairment to a cancer cell as compared to a non-cancer cell, adult stem cell, or post-mitotic cell.

### Embodiment 139

The method of any of embodiments 126-138, further comprising administering to the subject an antiviral agent configured to kill the flavivirus.

### Embodiment 140

The method of embodiment 139, wherein the antiviral agent comprises a nucleoside analog.

### Embodiment 141

The method of embodiment 140, wherein the nucleoside analog comprises sofosbuvir, 2′-C-methyladenosine, or a combination thereof.

### Embodiment 142

The method of any of embodiments 126-141, wherein the subject is administered an immunostimulatory agent.

### Embodiment 143

The method of embodiment 142, wherein the viral composition is administered in combination with the immunostimulatory agent.

### Embodiment 144

The method of embodiment 142, wherein the viral composition comprises the immunostimulatory agent.

### Embodiment 145

The method of any of embodiments 142-145, wherein the flavivirus or portion of the flavivirus targets the immunostimulatory agent to the cancer.

### Embodiment 146

The method of any of embodiments 142-145, wherein the viral composition and the immunostimulatory agent provide a synergistic immune response to the cancer.

### Embodiment 147

The method of embodiment of any of embodiments 142-146, wherein the viral composition and immunostimulatory agent are administered in a single composition.

### Embodiment 148

The method of any of embodiments 142-146, wherein the viral composition and immunostimulatory agent are administered as separate compositions.

### Embodiment 149

The method of any of embodiments 142-148, wherein the immunostimulatory agent comprises a Toll-like receptor (TLR) agonist.

### Embodiment 150

The method of embodiment 149, wherein the Toll-like receptor agonist comprises lipopolysaccharide (LPS), imiquimod (TLR7 agonist), rintatolimod, resiquimod, or a combination thereof.

### Embodiment 151

The method of any of embodiments 142-148, wherein the immunostimulatory agent comprises granulocyte-macrophage colony-stimulating factor (GMCSF), heat shock protein (Hsp) 70, Hsp90, Hsp110, CpG oligonucleotide, or a combination thereof.

### Embodiment 152

The method of any of embodiments 142-151, wherein the immunostimulatory agent comprises an adjuvant.

### Embodiment 153

A pharmaceutical composition comprising an effective amount of a flavivirus or portion of a flavivirus wherein the flavivirus is engineered to comprise a heterologous nucleic acid sequence encoding a suicide gene and a pharmaceutically acceptable excipient.

### Embodiment 154

The pharmaceutical composition of embodiment 153, wherein the flavivirus comprises Zika virus.

### Embodiment 155

The pharmaceutical composition of embodiment 153 or embodiment 154, wherein the suicide gene encodes a protein that converts a prodrug into a cytotoxic agent.

### Embodiment 156

The pharmaceutical composition of embodiment 155, wherein the protein that converts a prodrug into the cytotoxic agent has enzymatic activity selected from the group consisting of thymidine kinase activity, cytosine deaminase activity, purine nucleoside phosphorylase activity, uracil phosphoribosyl transferase activity, and thymidylate kinase activity.

What is claimed is:

1. A method of treating a brain cancer comprising administering to a subject in need thereof a viral composition comprising a flavivirus or portion of a flavivirus wherein the flavivirus is engineered to comprise a heterologous nucleic acid sequence encoding a suicide gene.

2. The method of claim 1, wherein the brain cancer is selected from the group consisting of astrocytoma, oligodendroglioma, ependymoma, meningioma, schwannoma, craniopharyngioma, germinoma, and pineocytoma.

3. The method of claim 1, wherein the flavivirus comprises Zika virus, spondweni virus, kedougous virus, or a combination thereof.

4. The method of claim 1, wherein the flavivirus comprises Zika virus.

5. The method of claim 1, wherein the flavivirus comprises a nucleic acid sequence encoding for a capsid protein of the flavivirus, a membrane protein of the flavivirus, an envelope protein of the flavivirus, a nonstructural (NS) protein of the flavivirus, or a combination thereof.

6. The method of claim 1, wherein the suicide gene is expressible under an inducible promoter.

7. The method of claim 6, wherein the inducible promoter is cytomegalovirus (CMV).

8. The method of claim 1, wherein the suicide gene encodes a protein that converts a prodrug into a cytotoxic agent.

9. The method of claim 8, wherein the protein that converts a prodrug into the cytotoxic agent has enzymatic activity selected from the group consisting of thymidine kinase activity, cytosine deaminase activity, purine nucleoside phosphorylase activity, uracil phosphoribosyl transferase activity, and thymidylate kinase activity.

10. The method of claim 8, wherein the prodrug is selected from the group consisting of ganciclovir, ganciclovir elaidic acid ester, penciclovir, acyclovir, valacyclovir, (E)-5-(2-bromovinyl)-2′deoxyuridine, zidovudine, 2′-exo-methanocarbathymidine, 5-fluorocytosine, 6-methylpurine deoxyriboside, fludarabine, 5-fluorocytosine, 5-fluorouracil, and azidothymidine.

11. The method of claim 8, wherein the prodrug is administered to the subject.

12. The method of claim 1, wherein the viral composition promotes an immune response to the brain cancer.

13. The method of claim 1, wherein the viral composition causes preferential mitotic impairment to a cancer cell as compared to a non-cancer cell, adult stem cell, or post-mitotic cell.

14. The method of claim 1, further comprising administering to the subject an antiviral agent configured to kill the flavivirus.

15. The method of claim 14, wherein the antiviral agent comprises a nucleoside analog.

16. The method of claim 15, wherein the nucleoside analog comprises sofosbuvir, 2′-C-methyladenosine, or a combination thereof.

17. The method of claim 1, wherein the subject is administered an immunostimulatory agent.

18. The method of claim 17, wherein the viral composition is administered in combination with the immunostimulatory agent.

19. The method of claim 18, wherein the viral composition comprises the immunostimulatory agent.

20. The method of claim 17, wherein the flavivirus or portion of the flavivirus targets the immunostimulatory agent to the cancer.

21. The method of claim 17, wherein the viral composition and the immunostimulatory agent provide a synergistic immune response to the cancer.

22. The method of claim 17, wherein the viral composition and immunostimulatory agent are administered in a single composition.

23. The method of claim 17, wherein the viral composition and immunostimulatory agent are administered as separate compositions.

24. The method of claim 17, wherein the immunostimulatory agent comprises a Toll-like receptor (TLR) agonist.

25. The method of claim 24, wherein the Toll-like receptor agonist comprises lipopolysaccharide (LPS), imiquimod (TLR7 agonist), rintatolimod, resiquimod, or a combination thereof.

26. The method of claim 17, wherein the immunostimulatory agent comprises granulocyte-macrophage colony-stimulating factor (GMCSF), heat shock protein (Hsp) 70, Hsp90, Hsp110, CpG oligonucleotide, or a combination thereof.

27. The method of claim 17, wherein the immunostimulatory agent comprises an adjuvant.

28. A pharmaceutical composition comprising an effective amount of a flavivirus or portion of a flavivirus wherein the flavivirus is engineered to comprise a heterologous nucleic acid sequence encoding a suicide gene and a pharmaceutically acceptable excipient.

29. The pharmaceutical composition of claim 28, wherein the flavivirus comprises Zika virus.

30. The pharmaceutical composition of claim 29, wherein the suicide gene encodes a protein that converts a prodrug into a cytotoxic agent.

31. The pharmaceutical composition of claim 30, wherein the protein that converts a prodrug into the cytotoxic agent has enzymatic activity selected from the group consisting of thymidine kinase activity, cytosine deaminase activity, purine nucleoside phosphorylase activity, uracil phosphoribosyl transferase activity, and thymidylate kinase activity.
